SEQUENCE LISTING (31 sequences)

SEQ ID NO: 1 - DNA, 591 bp, Artificial Sequence
  AAP from AAV Anc80
  misc_feature (1)..(3): n=CTG, ATG, ACG, TTG, GTG, ATT, ATA
nnngcgactcagagtcagtgcccgaccctcaaccactcggagaaccccccgcagccccctctggtgtgggatctaatacaatggctgcaggcggtggcgctccaatggcagacaataacgaaggcgccgacggagtgggtaacgcctcaggaaattggcattgcgattccacatggctgggcgacagagtcatcaccaccagcacccgaacctgggccctccccacctacaacaaccacctctacaagcaaatctccagccaatcgggaggcagcaccaacgacaacacctacttcggctacagcaccccctgggggtattttgactttaacagattccactgccacttctcaccacgtgactggcagcgactcatcaacaacaactggggattccggcccaagaagctcaacttcaagctcttcaacatccaggtcaaggaggtcacgacgaatgatggcaccacgaccatcgccaataaccttaccagcacggttcaggtctttacggactcggaataccagctcccgtacgtcctcggctctgcgcaccagggctgcctgcctccgttcccggcggacgtcttcatga

SEQ ID NO: 2 - protein, 196 aa, Artificial Sequence
  AAP from AAV Anc80
LATQSQCPTLNHSENPPQPPLVWDLIQWLQAVALQWQTITKAPTEWVTPQEIGIAIPHGWATESSPPAPEPGPSPPTTTTSTSKSPANREAAPTTTPTSATAPPGGILTLTDSTATSHHVTGSDSSTTTGDSGPRSSTSSSSTSRSRRSRRMMAPRPSPITLPARFRSLRTRNTSSRTSSALRTRAACLRSRRTSS

SEQ ID NO: 3 - DNA, 2667 bp, Artificial Sequence
  OptMin construct
ggacctttaattcaacccaacacaatatattatagttaaataagaattattatcaaatcatttgtatattaattaaaatactatactgtaaattacattttatttacaatcactcgacgaagacttgatcacccggggaagcgcgcgggattcctgccgccacggctgccgacggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagaggccggtagagcaatcaccccaggaaccagactcctcttcgggcatcggcaagaaaggccagcagcccgcgagaaagagactcaactttgggcagactggcgactcagagtcagtgcccgaccctcaaccactcggagaaccccccgcagccccctctggtgtgggatctaatacaatggctgcaggcggtggcgctccaatggcagacaataacgaaggcgccgacggagtgggtaacgcctcaggaaattggcattgcgattccacatggctgggcgacagagtcatcaccaccagcacccgaacctgggccctccccacctacaacaaccacctctacaagcaaatctccagccaatcgggaggcagcaccaacgacaacacctacttcggctacagcaccccctgggggtattttgactttaacagattccactgccacttctcaccacgtgactggcagcgactcatcaacaacaactggggattccggcccaagaagctcaacttcaagctcttcaacatccaggtcaaggaggtcacgacgaatgatggcaccacgaccatcgccaataaccttaccagcacggttcaggtctttacggactcggaataccagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggagtactttccttctcaaatgctgagaacgggcaacaactttcagttcagctacacgtttgaggacgtgccttttcacagcagctacgcgcacagccaaagcctggaccggctgatgaaccccctcatcgaccagtacctgtactacctgtctcggactcagaccacgagtggtaccgcaggaaatcggacgttgcaattttctcaggccgggcctagtagcatggcgaatcaggccaaaaactggctacccgggccctgctaccggcagcaacgcgtctccaagacaaccaatcaaaataacaacagcaactttgcctggaccggtgccaccaagtatcatctgaatggcagagactctctggtaaatcccggtcccgctatggcaacccacaaggacgacgaagacaaattttttccgatgagcggagtcttaatatttgggaaacagggagctggaaatagcaacgtggaccttgacaacgttatgataaccaacgaggaagaaattaaaaccaccaacccagtggccacagaagagtacggcacggtggccactaacctgcaatcggccaacaccgctcctgctacagggaccgtcaacagtcaaggagccttacctggcatggtctggcaggaccgggacgtgtacctgcagggtcctatctgggccaagattcctcacacggacggacactttcatccctcgccgctgatgggaggctttggactgaaacacccgcctcctcagatcctgattaagaatacacctgttcccgcgaatcctccaactaccttcagtccagctaagtttgcgtcgttcatcacgcagtacagcaccggacaggtcagcgtggaaattgaatgggagctgcagaaagaaaacagcaaacgctggaacccagagattcaatacacttccaactacaacaaatctacaaatgtggactttgctgttgacacaaatggcgtttattctgagcctcgccccatcggcacccgttacctcacccgtaatctgtaaactagcagctgatagcatgcggtaccgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattgggaccaatacgcccgcgtttcttccttttccccaccccaacccccaagttcgggtgaaggcccagggctcgcagccaacgtcggggcggcaagcc

SEQ ID NO: 4 - DNA, 2921 bp, Artificial Sequence
  IntronMin construct
ggacctttaattcaacccaacacaatatattatagttaaataagaattattatcaaatcatttgtatattaattaaaatactatactgtaaattacattttatttacaatcactcgacgaagacttgatcacccggggaagcgcgcgggattcctgccgccatggctgccgacggttatcttccaggtaagtactccctatcagtgatagagatctggacctttaattcaacccaacacaatatattatagttaaataagaattattatcaaatcatttgtatattaattaaaatactatactgtaaattacattttatttacaatcactcgacgaagacttgatcacccggggaagcgcgcgggattccaagggggagacctgtagtcagagcccccgggcagcacacactgacatccactcccttcctattgtttcagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagaggccggtagagcaatcaccccaggaaccagactcctcttcgggcatcggcaagaaaggccagcagcccgcgagaaagagactcaactttgggcagactggcgactcagagtcagtgcccgaccctcaaccactcggagaaccccccgcagccccctctggtgtgggatctaatacaatggctgcaggcggtggcgctccaatggcagacaataacgaaggcgccgacggagtgggtaacgcctcaggaaattggcattgcgattccacatggctgggcgacagagtcatcaccaccagcacccgaacctgggccctccccacctacaacaaccacctctacaagcaaatctccagccaatcgggaggcagcaccaacgacaacacctacttcggctacagcaccccctgggggtattttgactttaacagattccactgccacttctcaccacgtgactggcagcgactcatcaacaacaactggggattccggcccaagaagctcaacttcaagctcttcaacatccaggtcaaggaggtcacgacgaatgatggcaccacgaccatcgccaataaccttaccagcacggttcaggtctttacggactcggaataccagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggagtactttccttctcaaatgctgagaacgggcaacaactttcagttcagctacacgtttgaggacgtgccttttcacagcagctacgcgcacagccaaagcctggaccggctgatgaaccccctcatcgaccagtacctgtactacctgtctcggactcagaccacgagtggtaccgcaggaaatcggacgttgcaattttctcaggccgggcctagtagcatggcgaatcaggccaaaaactggctacccgggccctgctaccggcagcaacgcgtctccaagacaaccaatcaaaataacaacagcaactttgcctggaccggtgccaccaagtatcatctgaatggcagagactctctggtaaatcccggtcccgctatggcaacccacaaggacgacgaagacaaattttttccgatgagcggagtcttaatatttgggaaacagggagctggaaatagcaacgtggaccttgacaacgttatgataaccaacgaggaagaaattaaaaccaccaacccagtggccacagaagagtacggcacggtggccactaacctgcaatcggccaacaccgctcctgctacagggaccgtcaacagtcaaggagccttacctggcatggtctggcaggaccgggacgtgtacctgcagggtcctatctgggccaagattcctcacacggacggacactttcatccctcgccgctgatgggaggctttggactgaaacacccgcctcctcagatcctgattaagaatacacctgttcccgcgaatcctccaactaccttcagtccagctaagtttgcgtcgttcatcacgcagtacagcaccggacaggtcagcgtggaaattgaatgggagctgcagaaagaaaacagcaaacgctggaacccagagattcaatacacttccaactacaacaaatctacaaatgtggactttgctgttgacacaaatggcgtttattctgagcctcgccccatcggcacccgttacctcacccgtaatctgtaaactagcagctgatagcatgcggtaccgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattgggaccaatacgcccgcgtttcttccttttccccaccccaacccccaagttcgggtgaaggcccagggctcgcagccaacgtcggggcggcaagcc

SEQ ID NO: 5 - DNA, 21 bp, Artificial Sequence
  primer
aggtgaggctgcaaacagcta

SEQ ID NO: 6 - DNA, 25 bp, Artificial Sequence
  primer
tttctgagggatgaataaggcatag

SEQ ID NO: 7 - DNA, 25 bp, Artificial Sequence
  primer
tgcacattggcaacagcccctgatg

SEQ ID NO: 8 - DNA, 19 bp, Artificial Sequence
  primer
agtccgccctgagcaaaga

SEQ ID NO: 9 - DNA, 18 bp, Artificial Sequence
  primer
gcggtcacgaactccagc

SEQ ID NO: 10 - DNA, 25 bp, Artificial Sequence
  primer
caacgagaagcgcgatcacatggtc

SEQ ID NO: 11 - DNA, 21 bp, Artificial Sequence
  primer
attagcgtggcgtgcttttac

SEQ ID NO: 12 - DNA, 19 bp, Artificial Sequence
  primer
gggtcaggctcctctttgc

SEQ ID NO: 13 - DNA, 26 bp, Artificial Sequence
  probe
caaacacgcgcattaacgagagcacc

SEQ ID NO: 14 - DNA, 20 bp, Artificial Sequence
  primer
gccgaggacttgcatttctg

SEQ ID NO: 15 - DNA, 18 bp, Artificial Sequence
  primer
tcggccaaagccattctc

SEQ ID NO: 16 - DNA, 22 bp, Artificial Sequence
  probe
tccacgcgcaccttgcttcctc

SEQ ID NO: 17 - DNA, 21 bp, Artificial Sequence
  primer
ttctgcagctcccattcaatt

SEQ ID NO: 18 - DNA, 20 bp, Artificial Sequence
  primer
tcaaccagtcaaagctgaac

SEQ ID NO: 19 - DNA, 21 bp, Artificial Sequence
  probe
ccacgctgacctgtccggtgc

SEQ ID NO: 20 - DNA, 22 bp, Artificial Sequence
  primer
ccagtcacgacgttgtaaaacg

SEQ ID NO: 21 - DNA, 23 bp, Artificial Sequence
  primer
agcggataacaatttcacacagg

SEQ ID NO: 22 - DNA, 20 bp, Artificial Sequence
  primer
agccacctactcccaacatc

SEQ ID NO: 23 - DNA, 21 bp, Artificial Sequence
  primer
ggaacccctagtgatggagtt

SEQ ID NO: 24 - DNA, 16 bp, Artificial Sequence
  primer
cggcctcagtgagcga

SEQ ID NO: 25 - DNA, 21 bp, Artificial Sequence
  probe
cactccctctctgcgcgctcg

SEQ ID NO: 26 - DNA, 9298 bp, Artificial Sequence
  Donor plasmid comprising the OptMin construct
ggacctttaattcaacccaacacaatatattatagttaaataagaattattatcaaatcatttgtatattaattaaaatactatactgtaaattacattttatttacaatcactcgacgaagacttgatcacccggggaagcgcgcgggattcctgccgccacggctgccgacggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagaggccggtagagcaatcaccccaggaaccagactcctcttcgggcatcggcaagaaaggccagcagcccgcgagaaagagactcaactttgggcagactggcgactcagagtcagtgcccgaccctcaaccactcggagaaccccccgcagccccctctggtgtgggatctaatacaatggctgcaggcggtggcgctccaatggcagacaataacgaaggcgccgacggagtgggtaacgcctcaggaaattggcattgcgattccacatggctgggcgacagagtcatcaccaccagcacccgaacctgggccctccccacctacaacaaccacctctacaagcaaatctccagccaatcgggaggcagcaccaacgacaacacctacttcggctacagcaccccctgggggtattttgactttaacagattccactgccacttctcaccacgtgactggcagcgactcatcaacaacaactggggattccggcccaagaagctcaacttcaagctcttcaacatccaggtcaaggaggtcacgacgaatgatggcaccacgaccatcgccaataaccttaccagcacggttcaggtctttacggactcggaataccagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggagtactttccttctcaaatgctgagaacgggcaacaactttcagttcagctacacgtttgaggacgtgccttttcacagcagctacgcgcacagccaaagcctggaccggctgatgaaccccctcatcgaccagtacctgtactacctgtctcggactcagaccacgagtggtaccgcaggaaatcggacgttgcaattttctcaggccgggcctagtagcatggcgaatcaggccaaaaactggctacccgggccctgctaccggcagcaacgcgtctccaagacaaccaatcaaaataacaacagcaactttgcctggaccggtgccaccaagtatcatctgaatggcagagactctctggtaaatcccggtcccgctatggcaacccacaaggacgacgaagacaaattttttccgatgagcggagtcttaatatttgggaaacagggagctggaaatagcaacgtggaccttgacaacgttatgataaccaacgaggaagaaattaaaaccaccaacccagtggccacagaagagtacggcacggtggccactaacctgcaatcggccaacaccgctcctgctacagggaccgtcaacagtcaaggagccttacctggcatggtctggcaggaccgggacgtgtacctgcagggtcctatctgggccaagattcctcacacggacggacactttcatccctcgccgctgatgggaggctttggactgaaacacccgcctcctcagatcctgattaagaatacacctgttcccgcgaatcctccaactaccttcagtccagctaagtttgcgtcgttcatcacgcagtacagcaccggacaggtcagcgtggaaattgaatgggagctgcagaaagaaaacagcaaacgctggaacccagagattcaatacacttccaactacaacaaatctacaaatgtggactttgctgttgacacaaatggcgtttattctgagcctcgccccatcggcacccgttacctcacccgtaatctgtaaactagcagctgatagcatgcggtaccgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattgggaccaatacgcccgcgtttcttccttttccccaccccaacccccaagttcgggtgaaggcccagggctcgcagccaacgtcggggcggcaagccctgccatagccactacgggtacgtaggccaaccactagaactatagctagagtcctgggcgaacaaacgatgctcgccttccagaaaaccgaggatgcgaaccacttcatccggggtcagcaccaccggcaagcgccgcgacggccgaggtctaccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggctcaagcagtgatcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagtacttctcgacaagcttgtcgagactgcaggctctagagtcgacctgcaggcatgcaagcttgattgtcctcgagtttattgttcaaagatgcagtcatccaaatccacattgaccagatcgcaggcagtgcaagcgtctggcacctttcccatgatatgatgaatgtagcacagtttctgatacgcctttttgacgacagaaacgggttgagattctgacacgggaaagcactctaaacagtctttctgtccgtgagtgaagcagatatttgaattctgattcattctctcgcattgtctgcagggaaacagcatcagattcatgcccacgtgacgagaacatttgttttggtacctgtctgcgtagttgatcgaagcttccgcgtctgacgtcgatggctgcgcaactgactcgcgcacccgtttgggctcacttatatctgcgtcactgggggcgggtcttttcttggctccaccctttttgacgtagaattcatgctccacctcaaccacgtgatcctttgcccaccggaaaaagtctttgacttcctgcttggtgaccttcccaaagtcatgatccagacggcgggtgagttcaaatttgaacatccggtcttgcaacggctgctggtgttcgaaggtcgttgagttcccgtcaatcacggcgcacatgttggtgttggaggtgacgatcacgggagtcgggtctatctgggccgaggacttgcatttctggtccacgcgcaccttgcttcctccgagaatggctttggccgactccacgaccttggcggtcatcttcccctcctcccaccagatcaccatcttgtcgacacagtcgttgaagggaaagttctcattggtccagtttacgcacccgtagaagggcacagtgtgggctatggcctccgcgatgttggtcttcccggtagttgcaggcccaaacagccagatggtgttcctcttgccgaactttttcgtggcccatcccagaaagacggaagccgcatattggggatcgtacccgtttagttccaaaattttataaatccgattgctggaaatgtcctccacgggctgctggcccaccaggtagtcgggggcggttttagtcaggctcataatctttcccgcattgtccaaggcagccttgatttgggaccgcgagttggaggccgcattgaaggagatgtatgaggcctggtcctcctggatccactgcttctccgaggtaatccccttgtccacgagccacccgaccagctccatgtacctggctgaagtttttgatctgatcaccggcgcgtcagaattgggattctgattctctttgttctgctcctgcgtctgcgacacgtgcgtcagatgctgcgccaccaaccgtttacgctccgtgagattcaaacaggcgcttaaatactgttctaaattagtccacgcccactggagctcaggctgggttttggggagcaagtaattggggatgtagcactcgtccaccaccttgttcccgcctccggcgccgtttctggtctttgtgaccgcgaaccagtttggcaaagtcggctcgatcccgcggtaaattctctgaatcagtttttcgcgaatctgactcaggaaacgtcccaaaactaaggatttcaccccggtggtttccacgagcacgtgtaagtggaagtagctctctcccttctcaaattgcacaaagaaaagggcctccggggccttactcacacggcgccactccgtcagaaagtcgcgctgcagcttctcggccacggtcaggggtgcctgctcaatcagattcagatccaagtcagaatctggcggcaactcccactccttctcggccacccagttcacaaagctgtcagaaatgccgggcagatgctcgtcaaggtcgctggggaccttaatcacaatctcgtaaaaccccgccagggcggcagatccgcgcccgatggtgggacggtatgaataatccggaatatttataggtttttttattacaaaactgttacgaaaacagtaaaatacttatttatttgcgagatggttatcattttaattatctccatgatctattaatattccggagtatac

SEQ ID NO: 27 - DNA, 9552 bp, Artificial Sequence
  Donor plasmid comprising the IntronMin construct
ggacctttaattcaacccaacacaatatattatagttaaataagaattattatcaaatcatttgtatattaattaaaatactatactgtaaattacattttatttacaatcactcgacgaagacttgatcacccggggaagcgcgcgggattcctgccgccatggctgccgacggttatcttccaggtaagtactccctatcagtgatagagatctggacctttaattcaacccaacacaatatattatagttaaataagaattattatcaaatcatttgtatattaattaaaatactatactgtaaattacattttatttacaatcactcgacgaagacttgatcacccggggaagcgcgcgggattccaagggggagacctgtagtcagagcccccgggcagcacacactgacatccactcccttcctattgtttcagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagaggccggtagagcaatcaccccaggaaccagactcctcttcgggcatcggcaagaaaggccagcagcccgcgagaaagagactcaactttgggcagactggcgactcagagtcagtgcccgaccctcaaccactcggagaaccccccgcagccccctctggtgtgggatctaatacaatggctgcaggcggtggcgctccaatggcagacaataacgaaggcgccgacggagtgggtaacgcctcaggaaattggcattgcgattccacatggctgggcgacagagtcatcaccaccagcacccgaacctgggccctccccacctacaacaaccacctctacaagcaaatctccagccaatcgggaggcagcaccaacgacaacacctacttcggctacagcaccccctgggggtattttgactttaacagattccactgccacttctcaccacgtgactggcagcgactcatcaacaacaactggggattccggcccaagaagctcaacttcaagctcttcaacatccaggtcaaggaggtcacgacgaatgatggcaccacgaccatcgccaataaccttaccagcacggttcaggtctttacggactcggaataccagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggagtactttccttctcaaatgctgagaacgggcaacaactttcagttcagctacacgtttgaggacgtgccttttcacagcagctacgcgcacagccaaagcctggaccggctgatgaaccccctcatcgaccagtacctgtactacctgtctcggactcagaccacgagtggtaccgcaggaaatcggacgttgcaattttctcaggccgggcctagtagcatggcgaatcaggccaaaaactggctacccgggccctgctaccggcagcaacgcgtctccaagacaaccaatcaaaataacaacagcaactttgcctggaccggtgccaccaagtatcatctgaatggcagagactctctggtaaatcccggtcccgctatggcaacccacaaggacgacgaagacaaattttttccgatgagcggagtcttaatatttgggaaacagggagctggaaatagcaacgtggaccttgacaacgttatgataaccaacgaggaagaaattaaaaccaccaacccagtggccacagaagagtacggcacggtggccactaacctgcaatcggccaacaccgctcctgctacagggaccgtcaacagtcaaggagccttacctggcatggtctggcaggaccgggacgtgtacctgcagggtcctatctgggccaagattcctcacacggacggacactttcatccctcgccgctgatgggaggctttggactgaaacacccgcctcctcagatcctgattaagaatacacctgttcccgcgaatcctccaactaccttcagtccagctaagtttgcgtcgttcatcacgcagtacagcaccggacaggtcagcgtggaaattgaatgggagctgcagaaagaaaacagcaaacgctggaacccagagattcaatacacttccaactacaacaaatctacaaatgtggactttgctgttgacacaaatggcgtttattctgagcctcgccccatcggcacccgttacctcacccgtaatctgtaaactagcagctgatagcatgcggtaccgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattgggaccaatacgcccgcgtttcttccttttccccaccccaacccccaagttcgggtgaaggcccagggctcgcagccaacgtcggggcggcaagccctgccatagccactacgggtacgtaggccaaccactagaactatagctagagtcctgggcgaacaaacgatgctcgccttccagaaaaccgaggatgcgaaccacttcatccggggtcagcaccaccggcaagcgccgcgacggccgaggtctaccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggctcaagcagtgatcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagtacttctcgacaagcttgtcgagactgcaggctctagagtcgacctgcaggcatgcaagcttgattgtcctcgagtttattgttcaaagatgcagtcatccaaatccacattgaccagatcgcaggcagtgcaagcgtctggcacctttcccatgatatgatgaatgtagcacagtttctgatacgcctttttgacgacagaaacgggttgagattctgacacgggaaagcactctaaacagtctttctgtccgtgagtgaagcagatatttgaattctgattcattctctcgcattgtctgcagggaaacagcatcagattcatgcccacgtgacgagaacatttgttttggtacctgtctgcgtagttgatcgaagcttccgcgtctgacgtcgatggctgcgcaactgactcgcgcacccgtttgggctcacttatatctgcgtcactgggggcgggtcttttcttggctccaccctttttgacgtagaattcatgctccacctcaaccacgtgatcctttgcccaccggaaaaagtctttgacttcctgcttggtgaccttcccaaagtcatgatccagacggcgggtgagttcaaatttgaacatccggtcttgcaacggctgctggtgttcgaaggtcgttgagttcccgtcaatcacggcgcacatgttggtgttggaggtgacgatcacgggagtcgggtctatctgggccgaggacttgcatttctggtccacgcgcaccttgcttcctccgagaatggctttggccgactccacgaccttggcggtcatcttcccctcctcccaccagatcaccatcttgtcgacacagtcgttgaagggaaagttctcattggtccagtttacgcacccgtagaagggcacagtgtgggctatggcctccgcgatgttggtcttcccggtagttgcaggcccaaacagccagatggtgttcctcttgccgaactttttcgtggcccatcccagaaagacggaagccgcatattggggatcgtacccgtttagttccaaaattttataaatccgattgctggaaatgtcctccacgggctgctggcccaccaggtagtcgggggcggttttagtcaggctcataatctttcccgcattgtccaaggcagccttgatttgggaccgcgagttggaggccgcattgaaggagatgtatgaggcctggtcctcctggatccactgcttctccgaggtaatccccttgtccacgagccacccgaccagctccatgtacctggctgaagtttttgatctgatcaccggcgcgtcagaattgggattctgattctctttgttctgctcctgcgtctgcgacacgtgcgtcagatgctgcgccaccaaccgtttacgctccgtgagattcaaacaggcgcttaaatactgttctaaattagtccacgcccactggagctcaggctgggttttggggagcaagtaattggggatgtagcactcgtccaccaccttgttcccgcctccggcgccgtttctggtctttgtgaccgcgaaccagtttggcaaagtcggctcgatcccgcggtaaattctctgaatcagtttttcgcgaatctgactcaggaaacgtcccaaaactaaggatttcaccccggtggtttccacgagcacgtgtaagtggaagtagctctctcccttctcaaattgcacaaagaaaagggcctccggggccttactcacacggcgccactccgtcagaaagtcgcgctgcagcttctcggccacggtcaggggtgcctgctcaatcagattcagatccaagtcagaatctggcggcaactcccactccttctcggccacccagttcacaaagctgtcagaaatgccgggcagatgctcgtcaaggtcgctggggaccttaatcacaatctcgtaaaaccccgccagggcggcagatccgcgcccgatggtgggacggtatgaataatccggaatatttataggtttttttattacaaaactgttacgaaaacagtaaaatacttatttatttgcgagatggttatcattttaattatctccatgatctattaatattccggagtatac

SEQ ID NO: 28 - DNA, 8919 bp, Artificial Sequence
  pMB-GFP transgene-containing vector
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttccttgtagtctgcagtaacgattaacccgcgtttaaaccacgctacttattaagtagccaagctctagcggcctcggcctctgcataaataaaaaaaattagtcagccaagagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgagtatactcctgagaacttcagggtgagggatcctctaagggacccttgacgttttctttccccttcttttctacggttaagttcaagtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcccgaccggtcgcaaggcgcgccacaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcatagaacgccggtgaccagcaagtaggaaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccctaataaaggccgaaccacggggacgtggttcaattgcctttgaaaaacacgataataccatggccaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggctcgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgctggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaggtgtaaacgccgttaaccttactcgagatttctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacactagagcaaggcttctagaacgtagataagtaccatgcatagttcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaaggttggcttaattaaattacgcgtcgtcgcgattcagctgaagcatgctgaagtcgctttcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctactttttctctgtcacagaatgaaaatttttctgtcatctcttcgttattaatgtttgtaattgactgaatatcaacgcttatttgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggcctacgcttacttcgaatcacagctgagcttaagtcatagctagcacgac

SEQ ID NO: 29 - DNA, 8073 bp, Artificial Sequence
  pFB-GFP transgene-containing vector
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagcggcctcggcctctgcataaataaaaaaaattagtcagccatgagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgctctagaggatccaagcttatcgataccgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccggtaggccaaccactagaactatagctagagtcctgggcgaacaaacgatgctcgccttccagaaaaccgaggatgcgaaccacttcatccggggtcagcaccaccggcaagcgccgcgacggccgaggtctaccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaagtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggctcaagcagtgatcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagtacttctcgacaagcttgtcgagactgcaggctctagattcgaaagcggccgcgactagtgagctcgtcgacgtaggcatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcag

SEQ ID NO: 30 - DNA, 1244 bp, Artificial Sequence
  DNA fragment P10-CapAnc80start OPT
gtatacggacctttaattcaacccaacacaatatattatagttaaataagaattattatcaaatcatttgtatattaattaaaatactatactgtaaattacattttatttacaatcactcgacgaagacttgatcacccggggaagcgcgcgggattcctgccgccacggctgccgacggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagaggccggtagagcaatcaccccaggaaccagactcctcttcgggcatcggcaagaaaggccagcagcccgcgagaaagagactcaactttgggcagactggcgactcagagtcagtgcccgaccctcaaccactcggagaaccccccgcagccccctctggtgtgggatctaatacaatggctgcaggcggtggcgctccaatggcagacaataacgaaggcgccgacggagtgggtaacgcctcaggaaattggcattgcgattccacatggctgggcgacagagtcatcaccaccagcacccgaacctgggccctccccacctacaacaaccacctctacaagcaaatctccagccaatcgggaggcagcaccaacgacaacacctacttcggctacagcaccccctgggggtattttgactttaacagattccactgccacttctcaccacgtgactggcagcgactcatcaacaacaactggggattccggcccaagaagctcaacttcaagctcttcaacatccaggtcaaggaggtcacgacgaatgatggcaccacgaccatcgccaataaccttaccagcacggttcaggtctttacggactcggaataccagctcccgtacggctagcagctgatgcat

SEQ ID NO: 31 - DNA, 1498 bp, Artificial Sequence
  DNA Fragment P10-CapAnc80start IntronP10
gtatacggacctttaattcaacccaacacaatatattatagttaaataagaattattatcaaatcatttgtatattaattaaaatactatactgtaaattacattttatttacaatcactcgacgaagacttgatcacccggggaagcgcgcgggattcctgccgccatggctgccgacggttatcttccaggtaagtactccctatcagtgatagagatctggacctttaattcaacccaacacaatatattatagttaaataagaattattatcaaatcatttgtatattaattaaaatactatactgtaaattacattttatttacaatcactcgacgaagacttgatcacccggggaagcgcgcgggattccaagggggagacctgtagtcagagcccccgggcagcacacactgacatccactcccttcctattgtttcagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagaggccggtagagcaatcaccccaggaaccagactcctcttcgggcatcggcaagaaaggccagcagcccgcgagaaagagactcaactttgggcagactggcgactcagagtcagtgcccgaccctcaaccactcggagaaccccccgcagccccctctggtgtgggatctaatacaatggctgcaggcggtggcgctccaatggcagacaataacgaaggcgccgacggagtgggtaacgcctcaggaaattggcattgcgattccacatggctgggcgacagagtcatcaccaccagcacccgaacctgggccctccccacctacaacaaccacctctacaagcaaatctccagccaatcgggaggcagcaccaacgacaacacctacttcggctacagcaccccctgggggtattttgactttaacagattccactgccacttctcaccacgtgactggcagcgactcatcaacaacaactggggattccggcccaagaagctcaacttcaagctcttcaacatccaggtcaaggaggtcacgacgaatgatggcaccacgaccatcgccaataaccttaccagcacggttcaggtctttacggactcggaataccagctcccgtacggctagcagctgatgcat

(19) United States
(12) Patent Application Publication
(10) Pub. No.: US 2020/0231986 A1
(43) Pub. Date: Jul. 23, 2020

(54) PRODUCTION OF ADENO-ASSOCIATED VIRUSES IN INSECT CELLS

(71) Applicants: Massachusetts Eye and Ear Infirmary, Boston, MA (US); The Schepens Eye Research Institute, Inc., Boston, MA (US); Université de Nantes, Nantes (FR); Centre Hospitalier Universitaire de Nantes, Nantes (FR); Institut National de la Santé et de la Recherche Médicale, Paris (FR)

(72) Inventors: Eduard Ayuso, Nante (FR); Cecile Robin, St. Herblain (FR); Magalie Penaud-Budloo, Saint Sébastien sur Loire (FR); Achille Francois, Nantes (FR); Véronique Blouin, Sainte Luce Sur Loire (FR); Luk H. Vandenberghe, Weston, MA (US); Anna Claire Maurer, Boston, MA (US)

(21) Appl. No.: 16/650,035

(22) PCT Filed: Sep. 28, 2018
(86) PCT No.: PCT/US2018/053546

§ 371 Date: Mar. 24, 2020

Related U.S. Application Data
(60) Provisional application No. 62/565,851, filed on Sep. 29, 2017.

(51) Int. Cl. C12N 15/86; C07K 14/005

(52) U.S. Cl. CPC C12N 15/86; C07K 14/005; C12N 2750/14143; C12N 2710/14051; C12N 2750/14122; C12N 2710/14044

(57) ABSTRACT

This disclosure relates to the field of high scale production of recombinant Adeno-Associated Viruses (AAVs). The inventors have conceived of specific nucleic acid constructs that allow for high scale production of recombinant AAV particles in insect cells. Importantly, these nucleic constructs do not require the production of a heterologous AAP. This disclosure thus relates to a nucleic acid for producing AAV capsids in insect cells, where the nucleic acid includes a first open reading frame encoding the VP1, VP2, and VP3 proteins, and a second open reading frame encoding the Assembly-Activating Protein (AAP).

### FIELD OF THE INVENTION

The present invention relates to large-scale production of AAV particles, in particular, for their use in therapeutic methods.

### BACKGROUND

Adeno-associated viruses (AAV) are considered to be one of the most promising viral vectors for human gene therapy. AAV has the ability efficiently to infect dividing as well as non-dividing human cells, the AAV viral genome integrates into a single chromosomal site in the host cell's genome, and most importantly, even though AAV is present in many humans, it has never been associated with any disease.

Recombinant AAV for use in gene therapy has primarily been produced in mammalian cell lines such as, e.g., 293 cells, COS cells, HeLa cells, KB cells, and other mammalian cell lines (see, e.g., U.S. Pat. Nos. 6,156,303, 5,387,484, 5,741,683, 5,691,176, 5,688,676, US 20020081721, WO 00/47757, WO 00/24916, and WO 96/17947). However, in most of these mammalian cell culture systems, the number of AAV particles generated per cell is on the order of 10⁴ particles, and amplification of mammalian cells in suspension systems is challenging (see, e.g., Robert et al Biotechnol. J. 2017, 12, 1600193). For a clinical study, production of rAAV at an even larger scale is required. To overcome the problems of mammalian production systems, AAV production systems have been developed using insect cells (see, e.g., Urabe et al., 2002, Hum. Gene Ther. Vol. 13: 1935-1943; WO 2007/046703; Chen, 2008, Molecular Therapy, Vol. 16 (no 5): 924-930; Smith et al., 2009, Molecular Therapy, Vol. 11: 1888-1896; Mietzsch et al., 2014, 25 (no 3): 212-222; Mietzsch et al., 2015, Human Gene Ther, 26 (no 10): 688-697; US 2014/0127801). For production of AAV in insect cells from the baculovirus expression system, some modifications were necessary for production of the three AAV capsid proteins (VP1, VP2, and VP3) in the correct stoichiometry, as it was known that AAV particles containing reduced amounts of VP1 are less infectious.

In addition, as one would have predicted, the in vivo administration of a viral vector may induce a human immune response to foreign antigens. Immune responses may be directed against AAV vector components, or the transgene product, or both.

Animal models predicted many aspects of the human immune response toward the transgene product, but largely failed to predict responses to AAV capsid. Delineation of these responses, and crafting of strategies to circumvent or manage them, is critical to achieving clinical success with AAV vectors.

Because of the high degree of conservation in the amino acid sequence among AAV capsids, anti-AAV antibodies show cross-reactivity over a wide range of serotypes.

Thus, although antibodies to AAV2 are clearly the most prevalent in humans (up to 70%), which are the natural host for this serotype, antibodies recognizing virtually all AAV serotypes can be found in a large proportion of individuals. Among the most commonly used AAV vectors, antibodies to AAV5, carrying one of the least conserved capsid sequences, and to AAV8, are among the least prevalent.

Thus, consistent with current concepts in immunology, the human immune response to a vector may vary substantially depending on the tissue in which the vector is encountered, with outcomes ranging from unresponsiveness (e.g., gene transfer in the eye), to tolerance (e.g., to the transgene product following expression in the liver), to clearance of transduced cells (e.g., capsid T-cell responses in the liver).

There was thus a need to better understand the structure-function relationship of AAVs within the constraints of the particle architecture in order to modulate the pharmacology of this new class of drugs to improve transduction efficiency and specificity, alter tropism, and reduce immunogenicity. For these reasons, ancestral reconstruction methods to predict the amino acid sequence of putative ancestral AAV capsid monomers using maximum likelihood methods were performed by Zinn et al. (2015, Cell Reports, 12: 1056-1068).

Screening of the vector library that emerged from the resulting sequence space yielded a number of different ancestral AAV serotypes. These ancestral AAV serotypes were successfully produced in the HEK 293 cell line using an expressed auxiliary Assembly-Activating Protein (AAP) originating from an AAV2 (Zinn et al., 2015, Supra).

For high dose applications and eventual commercial products, however, scalable high yielding manufacturing methods for AAV are needed.

### SUMMARY OF THE INVENTION

This disclosure relates to non-naturally occurring nucleic acid molecules for producing capsids of an Adeno-Associated Virus (AAV) in insect cells, wherein the nucleic acid molecules include a first open reading frame encoding major capsid protein VP1, and minor capsid proteins VP2 and VP3, and a second open reading frame encoding the Assembly-Activating Protein (AAP).

In some embodiments, the open reading frame encoding an AAP functional in insect cells includes or consists of a start codon for translation selected from a group comprising CTG, ATG, ACG, TTG, GTG, ATT and ATA

In some embodiments, termed “Optmin,” the open reading frame encoding VP1, VP2, and VP3 includes or consists of a start codon for translation of the VP1 protein selected from a group that includes one or more of ACG, TTG, CTG, and GTG.

According to some aspects of the “Optmin” embodiments, the open reading frame encoding VP1, VP2, and VP3 proteins includes or consists of a start codon for translation of the VP2 protein selected from a group that includes once or more of ACG, TTG, CTG, and GTG.

In some embodiments, termed “IntronMin” herein, the open reading frame encoding the VP1, VP2, and VP3 proteins includes or consists of a synthetic intron sequence within the VP1-encoding sequence.

According to some aspects of the “IntronMin” embodiment, the nucleic acid further includes or consists of (i) a first expression control sequence controlling the expression of the VP1-encoding sequence and (ii) a second expression control sequence controlling the expression of the VP2 and VP3-encoding sequences.

According to other aspects of the “IntronMin” embodiment, the second regulatory sequence controlling the expression of the VP2 and VP3-encoding sequences is located in the intron sequence.

According to some aspects of the “IntronMin” embodiment, the open reading frame encoding the VP1, VP2, and VP3 proteins comprises a start codon for translation of the VP2 protein which is selected from a group that includes one or more of ACG, TTG, CTG and GTG

In some embodiments, nucleic acids for producing capsids of an Adeno-Associated Virus (AAV) in insect cells further include or consist of an expression cassette for expressing AAV Rep proteins.

This disclosure also relates to baculovirus vectors that include one or more nucleic acids for producing capsids of an AAV as described herein. The present disclosure further pertains to insect cells including a nucleic acid for producing capsids of an AAV as described herein or a baculovirus vector comprising such a nucleic acid.

In another aspect, this disclosure also relates to methods for producing AAV particles including a) culturing insect cells as described herein; and b) collecting the AAV particles produced by the insect cells cultured at step a). In some embodiments, these methods further include c) purifying the AAV particles collected at step b), which may consist of purifying the AAV particles by immunoaffinity chromatography, for example, by using a chromatography support allowing the purification of AAV8 particles (e.g., a chromatography support onto which an anti-AAV8 antibody or an AAV8-binding fragment thereof is immobilized).

The present disclosure also concerns methods of purifying AAV particles including the use of affinity chromatography with a chromatography support on which an anti-AAV8 antibody or an AAV8-binding fragment is immobilized.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF THE FIGURES

FIG. 1 is a schematic representation of the Anc80L65 “OptMin” nucleic acid construct.

FIG. 2 is a schematic representation of the Anc80L65 “IntronMin” nucleic acid construct.

FIG. 3A-3B are representations of Western blots of Sf9 cell extracts probed with anti-Rep polyclonal antibodies (FIG. 3A) and anti-Cap polyclonal antibodies (FIG. 3B).

FIG. 4 is a graph demonstrating the genetic stability (expressed in arbitray units) of different clones of the baculovirus OptMin construct (from left to right, BAC085-C1, BAC085-C2, BAC085-C3, BAC085-C4 and BAC085-05) generated in the bac-to-bac system. Constructs were transfected into Sf9 cells and examined in successive passages (Plp, P2, P3, P4, P5, P6, P7, P8, P9, and P10).

FIG. 5 is a graph demonstrating genetic stability (expressed in arbitray units) of baculovirus IntronMin construct (from left to right, BAC085-C1, BAC085-C2, BAC085-C3, BAC085-C4 and BAC085-05) generated in the bac-to-bac system. Constructs were transfected into Sf9 cells and examined in successive passages (Plp, P2, P3, P4, P5, P6, P7, P8, P9, P10).

FIG. 6 is a representation of a Western blot of Sf9 cell extracts probed with anti-VP polyclonal antibodies.

FIG. 7 is a representation of a Western blot showing detection of AAP from BEV Rep2CapAnc80_L65_OptMin and BEV Rep2CapAnc80_L65_IntronMin.

FIG. 8 is a schematic map of the baculovirus shuttle vector designated 664_Rep2CapAnc80L65_OPTmin, which includes an OptMin nucleic acid construct as depicted in FIG. 1.

FIG. 9 is a schematic map of the baculovirus shuttle vector designated 665_Rep2_CapAnc80L65_Intron, which includes an IntronMin nucleic acid construct as depicted in FIG. 2.

### DETAILED DESCRIPTION OF THE INVENTION

The present disclosure provides for materials and methods allowing a large scale production of purified AAV particles in insect cells.

The present inventors wished to design a simple, robust, and stable system for a high yield production of AAV particles (e.g., AAV-Anc80L65; see, e.g., Zinn et al., 2015, Cell Reports, 12:1056-1068). Further, the present inventors wished to produce AAV particles having good infectivity properties that can be used, for example, in gene therapy. In this context, the inventors have also conceived of a powerful method for purifying AAV particles, including those produced in insect cells, by methods described in the present specification. The AAV production system conceived by the inventors is mainly based on the specific design of nucleic acids that, when expressed in insect cells, lead to the formation of AAV capsid VP1, VP2 and VP3 proteins in a ratio allowing optimal structure of the capsid, and also imparting good infectious properties to the resulting AAV particles.

Further, as will be described in detail herein, the AAV production system in insect cells described herein does not require expression of auxiliary exogenous proteins for capsid formation, which contributes substantially to the robustness, the stability and the reproducibility of this production system.

As is shown in the Examples herein, the AAV production systems integrate the genetic material for a high yield capsid production. Notably, the AAV production system described herein does not require the production of an auxiliary AAP originating from another AAV for producing the AAV capsids.

To the best of the inventors' knowledge, it is shown for the first time herein that the putative AAP-encoding sequence of a AAV serotype allows for the production of a functional AAP protein.

### Definitions

The following definitions are provided to provide clarity with respect to the terms as they are used in the specification and claims.

Throughout the present specification and the accompanying claims, the words “comprise” and “include” and variations such as “comprises,” “comprising,” “includes,” and “including” are to be interpreted inclusively. In addition, the terms “comprise” and “include” encompass “consisting of” and “consisting essentially of.”

As used herein, the term “recombinant AAV” refers to an AAV genome in which at least one extraneous or heterologous polynucleotide is inserted into the naturally occurring AAV genome.

The phrase “recombinant AAV (rAAV) vectors” is used herein to denote vectors that are typically composed of, at a minimum, a transgene and a regulatory sequences, and 5′ and 3′ AAV inverted terminal repeats (ITRs). It is this recombinant AAV vector that is packaged into a capsid protein and delivered to a selected target cell.

As used herein, the term “vector” is a nucleic acid molecule that transfers and/or replicates an inserted nucleic acid molecule into and/or between host cells. In some embodiments, the vectors described herein are incapable of autonomous self-replication.

An “AAV viral particle” or “AAV vector particle” or “AAV particle” refers to a viral particle composed of the AAV capsid proteins VP1, VP2 and VP3 and, in some embodiments, also an encapsidated polynucleotide AAV vector.

As used herein, the term “heterologous” means derived from a genotypically distinct entity from that of the rest of the entity to which it is compared. For example, a polynucleotide introduced by genetic engineering techniques into a different cell type is a heterologous polynucleotide. When that polynucleotide is expressed, the polynucleotide can encode a heterologous polypeptide.

As used herein, the term “operably linked” refers to a juxtaposition wherein the components so described are in a relationship permitting them to function in their intended manner. For example, a promoter sequence is operably linked to a coding sequence if the promoter sequence drives transcription of the coding sequence. As another example, an intron sequence is operably linked to a transcriptional unit if the intron contains splice donor and splice acceptor sites allowing for proper splicing of the transcription unit. Operably linked means that the DNA sequences being linked are typically contiguous and, where necessary to join two protein coding regions, contiguous and in reading frame. However, since enhancers can function when separated from the promoter by several kilobases, and intronic sequences may be of variable length, some nucleotide sequences may be operably linked but not contiguous.

As used herein, the term “expression cassette” refers to a nucleic acid construct, generated recombinantly or synthetically, with a series of specified nucleic acid elements which permit transcription of a particular nucleic acid in a host cell. The expression cassette can be incorporated into a plasmid, chromosome, virus, or nucleic acid fragment.

As defined herein, a “nucleotide sequence” or a “nucleic acid” is intended to refer to a natural or synthetic linear and sequential array of nucleotides and/or nucleosides, and derivatives thereof. A nucleic acid may be in the form of RNA, such as mRNA or cRNA, or in the form of DNA, including, for instance, cDNA and genomic DNA, e.g., obtained by cloning or produced by chemical synthetic techniques or by a combination thereof. The DNA may be triple-stranded, double-stranded or single-stranded. Single-stranded DNA may be the coding strand, also known as the sense strand, or it may be the non-coding strand, also referred to as the anti-sense strand.

The term “nucleic acid construct” as used herein refers to a man-made nucleic molecule resulting from the use of recombinant DNA technology. A nucleic acid construct is a nucleic acid molecule, either single- or double-stranded, which has been modified to contain segments of nucleic acids that are combined and juxtaposed in a manner that would not otherwise exist in nature. In some embodiments, a nucleic acid construct may be integrated in a vector, such as in a plasmid, a bacmid or a baculovirus vector. In some embodiments, a nucleic acid construct may be integrated in the genome of a cell, such as in the genome of an insect cell.

“Packaging” as used herein refers to a series of subcellular events that result in the assembly and encapsulation of a viral vector, particularly an AAV vector. Thus, when a suitable vector is introduced into an insect cell under appropriate conditions, it can be assembled into a viral particle.

AAV “rep” and “cap” genes are genes encoding replication and encapsulation proteins, respectively. AAV rep and cap genes have been found in all AAV serotypes examined to date, and are described herein and in the references cited. The AAV cap gene, in accordance with the present disclosure, encodes a cap protein that is capable of packaging AAV vectors in the presence of rep and any necessary helper functions (from, for example, adenoviruses, herpes simplex viruses or baculoviruses) and is capable of binding target cellular receptors.

AAV “AAP” means an AAV Assembly-Activating Protein, which is required along with the VP1, VP2, and VP3 proteins for AAV capsid assembly.

“Expression control sequence” refers to a nucleic acid sequence that regulates the expression of a nucleotide sequence to which it is operably linked. An expression control sequence is “operably linked” to a nucleotide sequence when the expression control sequence controls and regulates the transcription and/or translation of the nucleotide sequence. Thus, an expression control sequence can include promoters, enhancers, internal ribosome entry sites (IRES), transcription terminators and splicing signal for introns. The term “expression control sequence” is intended to include, at a minimum, a sequence whose presence is designed to influence expression, and can also include additional advantageous components. It includes sequences or polyadenylation sequences (pA) which direct the addition of a polyA tail, i.e., a string of adenine residues at the 3′-end of a mRNA, sequences referred to as polyA sequences. It also can be designed to enhance mRNA stability. Expression control sequences that affect the transcription and translation stability, e.g., promoters, as well as sequences that effect the translation, e.g., Kozak sequences, are known in insect cells. Expression control sequences can be of such nature as to modulate the nucleotide sequence to which it is operably linked such that lower expression levels or higher expression levels are achieved.

As used herein, the term “promoter” or “transcription regulatory sequence” refers to a nucleic acid sequence that functions to control the transcription of one or more coding sequences, and is located upstream (with respect to the direction of transcription) of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active in most tissues under most physiological and developmental conditions. An “inducible” promoter is a promoter that is physiologically or developmentally regulated, e.g., by the application of a chemical inducer. A “tissue specific” promoter is active only in specific types of tissues or cells.

The term “enhancer,” as used herein, refers to a DNA sequence element to which transcription factors bind to increase gene transcription.

“Poly (A)” sites at the 3′ end of the transcript signal the addition of a series of adenines during the RNA processing step before migration to the cytoplasm. Poly(A) tails increase the stability of the RNA.

An “open reading frame” (ORF) is a contiguous and non-overlapping set of tri-nucleotide codons in DNA or RNA. An “open reading frame” is a reading frame that contains a start codon, the subsequent region, which usually has a length that is a multiple of 3 nucleotides, and ends with a stop codon.

In addition to an open reading frame beginning with a start codon close to its 5′ end, some further sequence requirements in the local environment of the start codon have to be fulfilled to initiate protein synthesis. One of these is the “Kozak sequence.” The amount of protein synthesized from a given mRNA is dependent on the strength of the Kozak sequence.

“Gene expression” is the process by which inheritable information from a gene, such as the DNA sequence, is made into a functional gene product, such as protein or nucleic acid. Thus, gene expression always includes transcription, but not necessarily translation into protein. rRNA and tRNA genes are an example for non-protein coding genes that are expressed into rRNA and tRNA, respectively, and not translated into protein. For gene expression to take place, a promoter has to be present near the gene to provide one or more binding sites and recruit one or more enzymes to start transcription.

The term “adeno-associated virus ITRs” or “AAV ITRs,” as used herein, refers to the inverted terminal repeats present at both ends of the DNA strand of the genome of an adeno-associated virus. The ITR sequences are required for efficient multiplication of the AAV genome. Another property of these sequences is their ability to form a hairpin. This characteristic contributes to AAVs self-priming which allows the primase-independent synthesis of the second DNA strand. The ITRs also have been shown to be required for integration of the wild-type AAV DNA into the host cell as well as for efficient encapsulation of the AAV DNA combined with generation of a fully assembled, DNAase-resistant AAV particles.

The composition of a transgene sequence of the rAAV vector will depend upon the use to which the resulting vector will be put. For example, one type of transgene sequence includes a reporter sequence, which, upon expression, produces a detectable signal. In another example, the transgene encodes a therapeutic protein or therapeutic functional RNA. In another example, the transgene encodes a protein or functional RNA that is intended to be used for research purposes, e.g., to create a somatic transgenic animal model harboring the transgene, e.g., to study the function of the transgene product. In another example, the transgene encodes a protein or functional RNA that is intended to be used to create an animal model of disease. Appropriate transgene coding sequences will be apparent to the skilled artisan.

The term “transduce” or “transduction,” as used herein, refers to the process whereby a foreign nucleotide sequence is introduced into a cell via a viral vector.

The term “transfection,” as used herein, refers to the introduction of DNA into a recipient eukaryotic cell, which encompasses an insect cell.

Surprisingly, the nucleic acid sequences of AAV, when engineered appropriately as described herein, are able to express a functional Assembly-Activating Protein (AAP) that contributes to the AAV capsid assembly process. Notably, the inventors have shown that, when the nucleic acid of the AAV is appropriately engineered to allow the expression of the AAV AAP, no heterologous AAP (e.g., an AAP originating from a distinct AAV such as AAV2) is required for the capsid assembly.

More precisely, the inventors have modified the nucleic acid encoding the VP1, VP2, and VP3 proteins of AAV (i.e., the cap gene of AAV) so as to generate a start codon functional in insect cells at the beginning of the open reading frame (ORF) encoding the putative Assembly-Activating Protein (AAP). The inventors have shown that the resulting expressed AAV AAP protein is fully functional in insect cells, since a correct capsid assembly of the AAV particles was obtained without requiring any trans-complementation by expression of a heterologous AAP protein (e.g., the expression of an AAP protein originating from a distinct AAV such as AAV2).

Since the start codon for translation of AAV AAP is located within the nucleic acid sequence that also encodes the AAV capsid proteins (i.e., the cap gene), and, more precisely, within the VP1-encoding sequence, the inventors have introduced a start codon for AAV AAP that does not simultaneously introduce a change in the amino acid sequence of the resulting AAV-VP1 protein.

As shown in the Examples herein, a nucleic acid encoding a functional AAP has been used successfully for producing AAV particles in insect cells. This feature of the AAV production system described herein allows for the production of AAV capsids without requiring the presence of a heterologous AAP, e.g., an AAP originating from a distinct AAV serotype.

The inventors have produced AAV particles by designing a nucleic acid allowing the expression of (i) AAV VP1, VP2 and VP3 proteins, respectively and (iii) the AAV AAP protein. In particular, the inventors have produced recombinant AAV particles and have shown that a transgene is effectively encapsidated within the AAV particles and that the resulting recombinant AAV particles possess infectivity properties and are able to effectively transduce target cells.

This disclosure relates to a nucleic acid for producing capsids of an Adeno-Associated Virus (AAV) in insect cells, wherein the nucleic acid comprises a first open reading frame encoding the VP1, VP2 and VP3 proteins, and a second open reading frame encoding the Assembly-Activating Protein (AAP).

Notably, the nucleic acid for producing capsids of an Adeno-Associated Virus (AAV) in insect cells leads to the the generation of virions composed of VP1, VP2, and VP3 in a stoeichiometry between 1:1:8 and 1:1:12, respectively, so as to promote the highest infectivity on a per particle basis.

In some embodiments, the start codon of the open reading frame encoding the Assembly-Activating Protein (AAP) is selected from a group of start codons that are functional in insect cells comprising CTG, CTG, ATG, ACG, TTG, GTG, ATT and ATA.

In some embodiments, the start codon of the open reading frame encoding the Assembly-Activating Protein (AAP) of the AAV is CTG, which was used in the nucleic acid constructs illustrated in the Examples herein.

In some embodiments, the open reading frame encoding the Assembly-Activating Protein (AAP) is the nucleic acid of SEQ ID NO:1. In some embodiments, the start codon at positions 1-3 of SEQ ID NO:1 is CTG.

According to a specific aspect, this disclosure relates to a nucleic acid encoding a functional AAP of the AAV, the nucleic acid being SEQ ID NO:1. In some embodiments, the start codon at positions 1-3 of SEQ ID NO:1 is CTG.

As used herein, the AAP encoded by the nucleic acid shown in SEQ ID NO:1 is the amino acid sequence of SEQ ID NO:2.

In some embodiments, the nucleic acid comprising an open reading frame encoding an Assembly-Activating Protein (AAP) in insect cells is the nucleic acid of SEQ ID NO:3, which also encodes the VP1, VP2 and VP3 capsid proteins (nucleic acid construct termed “OptMin” herein).

In some embodiments, the nucleic acid comprising an open reading frame encoding an Assembly-Activating Protein (AAP) in insect cells is the nucleic acid of SEQ ID NO:4, which also encodes the VP1, VP2 and VP3 capsid proteins (nucleic acid construct termed “IntronMin” herein).

Both nucleic acids of SEQ ID NO:3 and SEQ ID NO:4 allow the expression of the VP1, VP2 and VP3 proteins in insect cells, provided that the required AAV helper sequences are also provided in the insect cells, which helper sequences encompass expression cassette(s) encoding AAV Rep proteins.

As will be described in more detail herein, the inventors have designed two nucleic acid constructs, wherein each nucleic acid construct allows (i) the expression of the AAV VP1, VP2 and VP3 proteins and (ii) the expression of the AAV AAP protein, so as to effectively produce high titers of infectious AAV particles in appropriate insect cells, including so as to effectively produce high titers of infectious recombinant AAV particles containing a transgene-bearing nucleic acid in appropriate insect cells.

As will be detailed elsewhere in the present specification, the AAV particles according to the disclosure are produced in “appropriate” insect cells, which means insect cells that further express additional required genes for AAV capsid formation and transgene encapsulation (e.g., genes encoding AAV Rep proteins).

These nucleic acid constructs are termed (i) “OptMin” and (ii) “IntronMin” in the present specification and are described in more detail below.

Nucleic Acid Construct OptMin

According to some embodiments, the nucleic acid for producing capsids of an Adeno-Associated Virus (AAV) in insect cells comprises an open reading frame encoding the VP1, VP2 and VP3 proteins and further comprises an open reading frame encoding an Assembly-Activating Protein (AAP). According to some embodiments, the nucleic acid comprises an uninterrupted sequence encoding the VP1, VP2 and VP3 proteins and comprises three start codons for translation of each of the VP1-, VP2-, and VP3-encoding sequences, which start codons are all functional in insect cells.

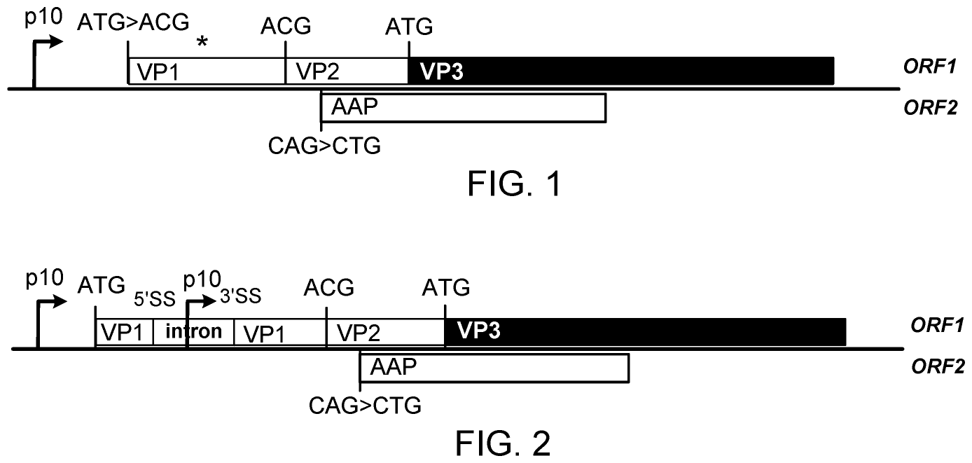

A schematic representation of the OptMin nucleic acid construct comprising the nucleic acid sequence is depicted in FIG. 1 herein.

In an OptMin nucleic acid construct, the start codons for translation of each of the VP1 and

VP2 open reading frames consist of start codons that are functionally suboptimal in insect cells, whereas the start codon for translation of the VP3 open reading frame functions as a strong start codon in insect cells. FIG. 1 shows that a start codon functional in insect cells was introduced for translation of the Assembly-Activating Protein (AAP). The asterisk in FIG. 1 indicates a silent mutation that removed an undesired potential start codon (ATG out of frame) located in the VP1 ORF. In the exemplary schematic shown in FIG. 1, a unique p10 promoter was located upstream of the VP1-coding sequence to drive the transcription and translation of VP1, VP2, VP3 and AAP.

Expression of the OptMin nucleic acid construct leads to the production of the AAV VP1, VP2 and VP3 proteins in ratios that allow for optimal AAV capsid assembly in insect cells, which leads to the production of infectious AAV particles in insect cells (e.g., AAV particles comprising one or more transgene-containing nucleic acid construct encapsidated therein).

Thus, in some embodiments of the OptMin nucleic acid construct, the start codon for translation of the VP1 protein is a suboptimal start codon in insect cells, wherein the start codon is selected from a group comprising or consisting of ACG, TTG, CTG, and GTG. In some embodiments, the start codon for translation of the VP1 protein is ACG.

In addition, in some embodiments of the OptMin nucleic acid construct, the start codon for translation of the VP2 protein is a suboptimal start codon in insect cells, the start codon being selected from a group comprising ACG, TTG, CTG and GTG. In some embodiments, the start codon for translation of the VP2 protein is ACG.

In some embodiments of the OptMin nucleic acid construct, the start codon for translation of the VP3 protein is a strong start codon, for example, the codon ATG.

According to some embodiments of the OptMin nucleic acid construct, one or more undesired strong start codons located in-frame or out-of-frame within any of the open reading frames encoding VP1, VP2 or VP3 may be removed by substitution of one or more nucleotides, provided that the nucleotide substitution does not cause a change in the corresponding encoded amino acid residue. Illustratively, an undesired ATG start codon located within the open reading frame encoding the AAV VP1 protein may be changed to an ACG codon without causing any change in the resulting amino acid sequence of the VP1 protein, as it is the case in the OptMin nucleic acid construct exemplified herein.

In some embodiments, the OptMin nucleic acid construct comprises, or consists of, the nucleic acid of SEQ ID NO:3.

In the OptMin construct comprising the sequence of SEQ ID NO:3, a CTG start codon for translation of the open reading frame encoding the AAV AAP protein has been introduced at the nucleotide positions 688-690 by replacing the initial nucleotide A at position 687 with the nucleotide T. It is specified that the introduction of this additional start codon, i.e. the introduction of a nucleotide substitution in a nucleic acid sequence that also encodes the AAV VP1, VP2 and VP3 proteins, does not cause any change in the amino acid sequence of the encoded VP1, VP2 and VP3 proteins.

In the OptMin construct comprising the nucleic acid of SEQ ID NO:3, a sub-optimal start codon for translation of the AAV VP1 has been introduced at the nucleotide positions 162-164. In some embodiments, the sub-optimal start codon for translation of the AAV VP1 is ACG.

In the OptMin construct comprising the nucleic acid of SEQ ID NO:3, a sub-optimal start codon for translation of the AAV VP2 is present at the nucleotide positions 573-575. In some embodiments, the sub-optimal start codon for translation of the AAV VP2 is ACG.

In the OptMin construct comprising the nucleic acid of SEQ ID NO:3, a strong start codon for translation of the AAV VP3 is present at the nucleotide positions 768-770. In some embodiments, the strong start codon for translation of the AAV VP3 is ATG.

Further, an undesirable strong start codon located out of frame within the open reading frame encoding VP1 of the OptMin construct of SEQ ID NO:3 (i.e., ATG) has been removed by replacing the nucleotide T at position 163 with the nucleotide C.

Without wishing to be bound by any particular theory, the inventors believe that the combination of sub-optimal start codons for translation of VP1 and VP2, respectively, and a strong start codon for translation of VP3, leads to the production of each of these proteins in respective amounts allowing an optimal AAV capsid assembly in insect cells, as well as good infectious properties of the resulting AAV particles. Further, as shown in the Examples herein, a nucleic acid construct comprising these start codon features is able to generate functional capsids of the AAV serotype wherein a transgene-containing nucleic acid construct may be encapsulated.

Highly surprisingly, the inventors have found that the OptMin construct allows the production of AAV particles encapsidating a transgene-containing construct at high yield in insect cells, the particles being infectious. Consequently, the OptMin construct allows the production of recombinant AAV particles for their use in gene therapy.

The inventors findings relating to the production of AAV particles in insect cells by using the OptMin nucleic acid construct are all the more surprising given that a similar type of nucleic acid construct failed to allow production of satisfactory AAV5 capsids, as described by Mietzsch et al. (2015, Human Gene Ther, Vol. 26 (no 10): 688-697). As shown by Mietzsch et al., such a construct did not allow production of a detectable level of VP1 from AAV5. The consequence was that the resulting AAV5 particles were endowed with a practically unquantifiable transduction efficiency, the resulting AAV5 particles being unsuitable for manufacturing recombinant AAVs for their use in methods of gene therapy.

In preferred embodiments of an OptMin nucleic acid construct, the nucleic acid sequence comprising the ORFs encoding the VP1, VP2, VP3 and AAP proteins, respectively, have not been engineered so as to be optimized according to the insect cell preferred codon usage. This lack of codon optimization of the sequence according to the insect cell preferred codon usage has permitted the inventors to avoid generating undesired additional start codons, and, thus, to avoid causing the translation of undesired proteins (e.g., truncated proteins) other than the expected VP1, VP2, VP3 and AAP proteins.

In some embodiments of the OptMin nucleic acid construct, the construct comprises an expression control sequence that drives the expression of the ORFs encoding VP1, VP2, VP3 and AAP proteins.

Thus, in some embodiments of the OptMin nucleic acid construct, the construct contains an expression cassette that comprises the open reading frames encoding VP1, VP2 and VP3 proteins and an expression control sequence functional in insect cells.

In some embodiments, the expression control sequence comprises a Kozak consensus sequence around each of the start codon for translation. Kozak consensus sequences are well known to one skilled in the art. The skilled person may refer to Chang et al. (1999, Virology, Vol. 259:369-393).

In some embodiments, the expression control sequence comprises a promoter sequence functional in insect cells. In some embodiments, the promoter may consist of a conditional promoter, either a repressible or an inducible promoter. In some other embodiments, the promoter is a constitutive promoter.

Techniques known to one skilled in the art for expressing foreign genes in insect host cells can be used to practice the methods described herein. Methodology for molecular engineering and expression of polypeptides in insect cells is described, for example, in Summers and Smith (1986, A Manual of Methods for Baculovirus Vectors and Insect Culture Procedures, Texas Agricultural Experimental Station Bull. No. 7555; College Station, Tex.; Luckow, 1991, In Prokop et al., Cloning and Expression of Heterologous Genes in Insect Cells with Baculovirus Vectors' Recombinant DNA Technology and Applications, 97-152; King and Possee, 1992, The baculovirus expression system, Chapman and Hall, United Kingdom; O'Reilly et al., 1992, Baculovirus Expression Vectors: A Laboratory Manual, New York; Freeman and Richardson, 1995, Baculovirus Expression Protocols, Methods in Molecular Biology, Vol 39; U.S. Pat. No. 4,745,051; US 2003/148506; and WO 03/074714). Suitable promoters for transcription of the ORFs described herein include, e.g., the polyhedrin (PoIH), p10, p35, IE-1 or AIE-1 promoters and further promoters described in the above references.

Promoters functional in insect cells can be selected from a group comprising or consisting of IE-1, polyhedrin, p10, and p35.

In some embodiments, the expression control sequence contained in the OptMin nucleic acid construct comprises one or more enhancer sequences. The enhancer element can be selected from hr1, hr2, hr3, hr4, and hr5.

In some embodiments, the OptMin nucleic acid construct also comprises a polyadenylation sequence. Polyadenylation sequences are well known to those skilled in the art.

In the OptMin construct illustrated in the Examples herein, the open reading frames encoding VP1, VP2, VP3 and AAP, respectively, are operably linked to the p10 constitutive strong promoter. Regarding the p10 promoter, one skilled in the art may refer to Knebel et al. (1985, Embo. J., Vol. 4 (5):1301-1306).

In some embodiments of the OptMin nucleic acid construct, the construct is shown in SEQ ID NO:3.

In the Optmin nucleic acid construct of SEQ ID NO:3, the p10 promoter sequence starts at position 1 and ends at position 155.

The Optmin nucleic acid construct of SEQ ID NO:3 can comprise a Kozak consensus sequence around the start codon of the VP1-encoding sequence, which Kozak consensus sequence can start at position 156 and end at position 165.

In the Optmin nucleic acid construct of SEQ ID NO:3, the open reading frame encoding the VP1, VP2 and VP3 proteins starts at position 162 and ends at position 2372. The sequence encoding VP1 starts at position 162 and ends at position 2372. The sequence encoding VP2 starts at position 573 and ends at position 2372. The sequence encoding VP3 starts at position 768 and ends at position 2372. The sequence encoding AAP starts at position 688 and ends at position 1278.

The AAV VP1 protein is encoded by the sequence starting at position 162 and ending at position 2372 of SEQ ID NO:3. The AAV VP2 protein is encoded by the sequence starting at position 273 and ending at position 2372 of SEQ ID NO:3. The AAV VP3 protein is encoded by the sequence starting at position 768 and ending at position 2372 of SEQ ID NO:3.

In the OptMin nucleic acid construct of SEQ ID NO:3, a polyadenylation signal is present. More precisely, the nucleic acid construct of SEQ ID NO:3 comprises a polyadenylation signal from the Herpes simplex virus type 1 thymidine kinase (also termed HSV-tk), which starts at position 2404 and ends at position 2667.

In some embodiments, an OptMin nucleic acid construct as described herein is included in a vector that is functional in insect cells, and typically included in a baculovirus vector, as will be described elsewhere in the present specification.

Nucleic Acid Construct IntronMin

According to some other embodiments of the nucleic for expressing the VP1, VP2 and VP3 proteins of an Adeno-Associated Virus (AAV) in insect cells, wherein the nucleic acid comprises an open reading frame encoding the VP2 and VP3 proteins comprises a synthetic intron sequence within the VP1-encoding sequence. Indeed, the synthetic intron sequence is functional in insect cells.

The synthetic intron may also be termed “heterologous intron” or “exogenous intron” or simply “intron” in the present specification, wherein is is understood that the intron is functional in insect cells.

A schematic representation of the IntronMin nucleic acid construct comprising the nucleic acid sequence is depicted in FIG. 2 herein.

In FIG. 2, a strong start codon is present for translation of VP1, and the VP1 ORF comprises a synthetic intronic sequence that is functional in insect cells. The start codon for translation of VP2 is sub-optimal in insect cells, and the start codon for translation of VP3 is a strong start codon. A start codon functional in insect cells has been introduced for translation of the AAV Assembly-Activating Protein (AAP). A first p10 promoter located upstream the VP1-coding sequence drives the translation of VP1, VP2, VP3 and AAP, and a second p10 promoter located in the synthetic intronic sequence and upstream of the VP2-coding sequence drives the translation of VP2, VP3 and AAP.

As disclosed in the Examples, such nucleic acid comprises an inserted exogenous intron sequence located within the open reading frame encoding the AAV VP1 protein, the exogenous intron being located upstream of the VP2 and VP3 open reading frames.

Thus, the transcription of the nucleic acid comprised in the IntronMin construct in insect cells generates two mRNAs, (i) a first mRNA comprising the open reading frames encoding the AAV VP1, VP2 and VP3 proteins and (ii) a second mRNA comprising the open reading frames encoding the AAV VP2 and VP3 proteins. In addition, both the first and second mRNAs also encode the AAV AAP protein.

In an IntronMin nucleic acid construct, the start codon for translation of each of the VP1 open reading frames is a strong start codon. Also in an IntronMin nucleic acid construct, the start codon for translation of VP2 is a sub-optimal start codon and the start codon for translation of VP3 is a strong start codon.

Without wishing to be bound by any particular theory, the inventors believe that the first mRNA comprising the open reading frames encoding the AAV VP1, VP2, VP3, and AAP proteins in insect cells leads mainly to the translation of the VP1 and the AAP sequences, because the start codon of the VP1 coding sequence consists of a strong start codon and most of the ribosomes will recognize the strong start codon for VP1 and few ribosomes will reach the start codons for VP2 and VP3, respectively. Thus, the inventors believe that the VP2 and VP3 proteins are produced mainly by translation of the second mRNA comprising the open reading frames encoding the AAV VP2, VP3 and AAP proteins.

Thus, expression of the IntronMin nucleic acid construct leads to the production of the AAV VP1, VP2 and VP3 proteins in ratios allowing an optimal AAV capsid assembly in insect cells, and the expression leads to the production of infectious AAV particles in insect cells.

In some embodiments of the IntronMin nucleic acid construct, the start codon for translation of VP1 is ATG.

In some embodiments of the IntronMin nucleic acid construct, the start codon for translation of the VP2 protein is a sub-optimal start codon in insect cells selected from a group comprising or consisting of ACG, TTG, CTG, and GTG.

In some embodiments of the IntronMin nucleic acid construct, the start codon for translation of VP3 is ATG.

According to other embodiments of the IntronMin nucleic acid construct, one or more undesired strong start codons located in-frame or out-of-frame with any of the open reading frames encoding VP1, VP2, or VP3 can be removed by substitution of a nucleotide, provided that the nucleotide substitution does not cause a change in the corresponding encoded amino acid residue. Illustratively, an undesired ATG start codon located within the open reading frame encoding the AAV VP1 protein can be changed to an ACG codon, as is the case for the IntronMin nucleic acid construct exemplified herein. In some embodiments, the IntronMin nucleic acid construct comprises, or consists of, SEQ ID NO:4.

In the IntronMin construct comprising the sequence of SEQ ID NO:4, a CTG start codon for translation of the open reading frame encoding the AAV AAP protein has been introduced at nucleotide positions 942-944 by replacing the initial nucleotide A at position 943 with the nucleotide T. The introduction of this additional start codon, i.e., the introduction of a nucleotide substitution in a nucleic acid sequence that also encodes the AAV VP1, VP2, and VP3 proteins, does not cause any change in the amino acid sequence of the thus encoded capsid proteins.

In the IntronMin construct of SEQ ID NO:4, a strong start codon for translation of the AAV VP1 is present at the nucleotide positions 162-164. In some embodiments, ATG is the strong start codon for VP1.

In the IntronMin construct comprising the nucleic acid of SEQ ID NO:4, a sub-optimal start codon for translation of the AAV VP2 is present at nucleotide positions 827-829. In some embodiments, ACG is the sub-optimal start codon for VP2.

In the IntronMin construct comprising the nucleic acid of SEQ ID NO:4, a strong start codon for translation of the AAV VP3 is present at nucleotide positions 1022-1024. In some embodiments, ATG is the strong start codon for VP3.

Further, an undesirable strong start codon (i.e., ATG) located within the open reading frame encoding VP1 of the IntronMin construct of SEQ ID NO:4 has been deleted by replacing the initial nucleotide T at position 173 with the nucleotide C.

In the IntronMin construct, the intronic sequence starts at position 187 and ends at position 440 of SEQ ID NO:4.

Without wishing to be bound by any particular theory, the inventors believe that the combination of (i) the generation of distinct mRNAs for VP1 and VP2/VP3, respectively, (ii) the presence of a sub-optimal start codon for translation for VP2, and (iii) the presence of a strong start codon for translation of VP3, as well as a functional open reading frame encoding the AAP protein, lead to the production of each of the capsid proteins in respective amounts allowing an optimal capsid assembly, good encapsulation of a transgene-containing construct, as well as good infectious properties of the resulting AAV particles.

Highly surprisingly, the inventors have found that the IntronMin construct allows the production of AAV particles encapsulating a transgene-containing construct at high yield in insect cells, with the particles being infectious. Consequently, the IntronMin construct allows the production of recombinant AAV particles for their use in gene therapy.

In some embodiments of an IntronMin nucleic acid construct, the nucleic acid sequence comprising the ORFs encoding the VP1, VP2, VP3 and AAP proteins has not been engineered so as to be optimized according to the insect cell preferred codon usage. This lack of codon optimization of the sequence according to the insect cell preferred codon usage has permitted the inventors to avoid generation of undesired additional start codons, and thus to avoid translation of truncated proteins other than the expected VP1, VP2, VP3 and AAP proteins.

In some embodiments of the IntronMin nucleic acid construct, the construct comprises a first expression control sequence that drives the expression of the ORF encoding the VP1, VP2 and VP3 proteins as well as the AAP protein. In some embodiments of the IntronMin nucleic acid construct, the construct comprises a second expression control sequence that drives the expression of the ORF encoding VP2 and VP3 proteins as well as of the AAP protein. In some embodiments, the second expression control sequence is located in the intronic sequence.

Consequently, according to the IntronMin nucleic acid construct, two distinct transcripts (i) VP1, VP2, VP3, and AAP and (ii) VP2, VP3 and AAP, respectively, are generated. Thus, translation of AAP is effected from both transcripts.

In some embodiments, the expression control sequence upstream of the VP1-encoding sequence comprises a Kozak consensus sequence. Kozak consensus sequences are well known to those skilled in the art. A skilled person may refer to Chang et al. (1999, Virology, Vol. 259: 369-393).

In some embodiments, the first expression control sequence controlling the expression of VP1, VP2, VP3, and AAP and the second expression control sequence controlling the expression of VP2, VP3, and AAP are the same. In some embodiments, the first expression control sequence controlling the expression of VP1, VP2, VP3, and AAP and the second expression control sequence controlling the expression of VP2, VP3, and AAP are different.

In some embodiments, the expression control sequences comprise, or consist of, promoter sequences functional in insect cells. Promoters functional in insect cells can be selected from a group comprising IE-1, polyhedrin, p10, or p35.

In some embodiments, the promoter is a conditional promoter (e.g., a repressible or an inducible promoter). In some embodiments, the promoter is a constitutive promoter.

In some embodiments, the expression control sequence contained in the IntronMin nucleic acid construct comprises one or more enhancer sequences. In some embodiments, the enhancer element is selected from the group consisting of hr1, hr2, hr3, hr4, and hr5.

In some embodiments, the IntronMin nucleic acid construct also comprises a polyadenylation sequence. Polyadenylation sequences are well known to one skilled in the art.

In the IntronMin construct, which is illustrated in the Examples herein, the open reading frames encoding VP1, VP2, VP3, and AAP are operably linked to a first p10 constitutive strong promoter, located upstream of the VP1 open reading frame.

In the IntronMin construct which is illustrated in the Examples herein, the open reading frames encoding VP2, VP3, and AAP are operably linked to a second p10 constitutive strong promoter, which is located upstream of the open reading frame encoding VP2 in the intronic sequence present within the VP1 open reading frame.

Regarding the p10 promoter, one skilled in the art may refer to Knebel et al. (1985, Embo J, Vol. 4 (no 5): 1301-1306).

In some embodiments of the IntronMin nucleic acid construct, the construct has the sequence shown in SEQ ID NO:4.

In the IntronMin nucleic acid construct of SEQ ID NO:4, the first p10 promoter sequence controlling the expression of VP1, VP2, VP3, and AAP starts at position 1 and ends at position 155.

In the IntronMin nucleic acid construct of SEQ ID NO:4, the synthetic intronic sequence starts at the nucleotide at position 187 and ends at position 440.

In the IntronMin nucleic acid construct of SEQ ID NO:4, the second p10 promoter sequence controlling the expression of VP2, VP3 and AAP starts at position 217 and ends at position 370.

The IntronMin nucleic acid construct of SEQ ID NO:4 comprises a Kozak consensus sequence around the start codon of the VP1-encoding sequence, which starts at position 156 and ends at position 165.

In the IntronMin nucleic acid construct of SEQ ID NO:4, the open reading frame encoding the VP1 protein starts at position 162 and ends at position 2626 and comprises an intronic sequence that starts at position 187 and ends at position 440. Otherwise, the open reading frame encoding the VP1 protein corresponds to positions 162-186 and 441-2626 of SEQ ID NO:4. In the IntronMin nucleic acid construct of SEQ ID NO:4, the open reading frame encoding the VP2 and VP3 proteins starts at position 827 and ends at position 2626. The sequence encoding VP2 starts at position 827 and ends at position 2626. The sequence encoding VP3 starts at position 1022 and ends at position 2626. The sequence encoding AAP starts at position 942 and ends at position 1532.

In the OptMin nucleic acid construct of SEQ ID NO:4, a polyadenylation signal is present. More precisely, the nucleic acid construct of SEQ ID NO:4 comprises a polyadenylation signal from the Herpes simplex virus type 1 thymidine kinase (also termed HSV-tk), which starts at position 2658 and ends at position 2921.

In some embodiments, an IntronMin nucleic acid construct as described herein is included in a vector that is functional in insect cells (e.g., a baculovirus vector), as will be further described in the present specification.

Insect Cells or Vectors Comprising an Optmin or an IntronMin Construct

In some embodiments, the OptMin construct or the IntronMin construct is comprised in a vector which is functional in insect cells, for example, a baculovirus vector.

Such a vector functional in insect cells is understood to be a nucleic acid molecule capable of productive transformation or transfection of an insect or insect cell. Exemplary biological vectors include plasmids, linear nucleic acid molecules, and recombinant viruses. Any vector can be employed as long as it is functional in insect cells.

The vector may integrate into the genome of the insect cells but the vector may also be episomal. The presence of the vector in the insect cell need not be permanent, and transient episomal vectors are also encompassed herein.

The vectors may be introduced by any means known, for example by chemical treatment of the cells, by electroporation, or by infection.

In some embodiments, the vector is a baculovirus, i.e. the OptMin construct or the IntronMin construct is comprised in a baculovirus vector. Baculovirus vectors and methods for their use are well known to one skilled in the art.

The number of nucleic acid vectors employed in the insect cell for the production of AAV particles, including recombinant AAV particles, is not limiting. For example, one, two, three or more separate vectors can be employed to produce AAV particles in insect cells in accordance with known methods.

If three vectors are used, a first vector can include the OptMin construct or the IntronMin construct, a second vector can include a nucleic acid construct encoding the AAV Rep proteins and a third vector can include at least one AAV inverted terminal repeat (ITR).

If two vectors are used, a first vector can include (i) the OptMin construct or the IntronMin construct and (ii) a nucleic acid construct encoding the Rep proteins, and a second vector can include at least one AAV ITR.

Nucleic acid constructs comprising expression cassettes for AAV Rep proteins in insect cells, and especially baculovirus vectors comprising the expression cassettes, are well known in the art. The one skilled in the art may refer to US 2014/0127801, Urabe et al. (2002, Human Gene Therapy, Vol. 13: 1935-1943), Urabe et al. (2006, J Virology, Vol. 80 (no 4): 1874-1885); Chen (2008, Molecular Therapy, Vol. 16 (no 5): 924-930), Smith et al. (2009, Molecular Therapy, Vol. 17 (no 11): 1888-1896), Aslanidi et al. (2009, Proc Natl Acad Sci, Vol. 106 (no 13): 5059-5064); Mietzsch et al. (2014, Vol. 25 (no 3): 212-222) and Mietzsch et al. (2015, Human Gene Therapy, Vol. 26 (10):688-697).

According to the present disclosure, the nucleic acid sequences encoding the Rep proteins encompass sequences encoding the Rep proteins originating from any known AAV serotype. Thus, Rep protein-coding nucleic acid sequences can be from any of the naturally occurring AAV serotypes, including, but not limited to, AAV1, AAV2, AAV3, AAV4, AAV5, AAV6, AAV7, AAV8, and AAV9, or variants thereof.

In some embodiments, the nucleic acid sequences encoding the AAV Rep proteins originate from AAV2. For appropriate constructs encoding the Rep proteins originating from AAV2, one skilled in the art may refer to Smith et al. (2009, Molecular Therapy, Vol. 17 (11):1888-1896), and especially to the Materials and Methods section on page 1894 thereof that describes the “Plasmid and recombinant baculovirus construction.”

In some embodiments, the nucleic acid construct for expressing the Rep proteins includes one or more expression cassettes for expressing Rep78 and Rep52. In some embodiments, the nucleic acid construct includes an open reading frame encoding both Rep78 and Rep52, and (i) the start codon for translation of the Rep78 is a sub-optimal start codon in insect cells and (ii) the start codon for translation of the Rep52 is a strong start codon in insect cells.

Thus, in some embodiments, the start codon for translation of Rep78 is selected from a group comprising CTG, ACG, TTG, GTG, ATT, and ATA. In some embodiments, the start codon for translation of the Rep52 is ATG.

In some embodiments, the nucleic acid construct OptMin or IntronMin and the nucleic acid construct for expressing Rep proteins are both integrated in the genome of the insect host cells which are designed for producing AAV particles.

One of ordinary skill in the art knows how to stably introduce a nucleotide sequence into the insect genome and how to identify a cell having such a nucleotide sequence in the genome (see, e.g., Aslanidi et al, (2009) PNAS, 106: 5059-5064). The incorporation into the genome may be aided by, for example, the use of a vector comprising nucleotide sequences highly homologous to regions of the insect genome. The use of sequences such as transposons is another way to introduce a nucleotide sequence into a genome.

In some embodiments, (i) the nucleic acid construct for expressing Rep proteins is integrated in the genome of the insect host cells and (ii) the nucleic acid construct OptMin or IntronMin is located in an appropriate vector, for example, in a baculovirus vector.

In some embodiments, (i) the nucleic acid construct OptMin or IntronMin is located in an appropriate vector, for example, in a baculovirus vector and (ii) the nucleic acid construct for expressing Rep proteins is integrated in the genome of the insect host cells.

In some embodiments, the nucleic acid construct OptMin or IntronMin and the nucleic acid construct for expressing Rep proteins are located in distinct nucleic acid vectors, such as in distinct baculovirus vectors.

Thus, in some embodiments, (i) the OptMin construct or the IntronMin construct and (ii) the nucleic acid construct comprising the expression cassettes for the AAV Rep proteins are located in separate vectors, e.g., in separate baculovirus vectors.

In some embodiments, (i) the OptMin construct or the IntronMin construct and (ii) the nucleic acid construct comprising the expression cassettes for the AAV Rep proteins are located within the same nucleic acid vector, e.g., within the same baculovirus vector. These embodiments are illustrated in the Examples herein.

Expression cassettes for the production of AAV Rep proteins in insect cells can be selected from nucleic acid sequences encoding both Rep78 and Rep52 or nucleic acid sequences encoding both Rep68 and Rep40.

In some embodiments, the nucleic acid construct for the AAV Rep proteins comprises a nucleic acid sequence encoding both Rep78 and Rep52, and the nucleic acid comprising a sub-optimal start codon for translation of Rep78 and a strong start codon for translation of Rep52.

In some embodiments, the open reading frame encoding Rep78/Rep52 is operably linked to a strong constitutive promoter functional in insect cells. Such promoters are described elsewhere in the present specification. In illustrative embodiments, the promoter is the polyhedrin promoter, polh.

In some embodiments, the AAV particles, which are produced according to the present disclosure, consist of recombinant AAV particles that comprise an encapsidated transgene-containing nucleic acid construct.

The transgene-containing nucleic acid construct is expressed in the insect host cells that also express (i) the OptMin or the IntronMin construct as well as (ii) the Rep construct(s), the expressed transgene-containing nucleic acid construct being encapsidated in the AAV particles that are formed within the insect host cells.

Thus, in some embodiments, the AAV described herein are recombinant AAV, a further nucleic acid construct is present in the insect cells that comprises a nucleic acid encoding a transgene of interest and at least one or two AAV-derived ITR sequence(s). As is known in the art, the ITR sequences cause encapsulation of the transgene-encoding nucleic acid construct within the AAV capsids that are formed in the insect host cells.

In some embodiments, the transgene-encoding nucleic acid is located in the transgene-encoding nucleic acid construct between two AAV-derived ITR sequences.

The ITR sequences can be any ITR sequence known to one skilled in the art to be effective for encapsulation in an AAV particle. The ITR sequences may originate from a naturally occurring AAV serotype comprising, but not limited to, AAV1, AAV2, AAV3, AAV4, AAV5, AAV6, AAV7, AAV8 and AAV9 or variants thereof. Illustratively, the ITR sequences may originate from an AAV2, as shown in the Examples herein.

In some embodiments, the transgene nucleic acid consists of a nucleic acid whose expression in mammalian cells (e.g., human cells) is desired. The nucleic acid may encode a nucleic acid of interest (e.g. a RNAi, a ribozyme, a miRNA, etc.) or may encode a polypeptide of interest (e.g., a protein ligand, a therapeutic protein, an antibody, etc.).

Any nucleotide sequence can be incorporated for later expression in a mammalian cell transfected with the recombinant AAV particles produced in insect cells.

In some embodiments, in the transgene-encoding construct, the nucleic acid whose expression in mammalian cells is desired can be operably linked to at least one expression control sequence that is functional in mammalian cells.

In some embodiments, the transgene-encoding nucleic acid construct is integrated within the genome of the insect host cell.

In some embodiments, the transgene-encoding nucleic acid construct is integrated in an appropriate vector functional in insect cells, such as a baculovirus vector.

This disclosure also relates to a recombinant insect cell that has been transfected by, or that has been transformed with, an OptMin nucleic acid construct as described herein.

This disclosure further relates to a recombinant insect cell that has been transfected by, or that has been transformed with, an IntronMin nucleic acid construct as described herein.

In some embodiments, the recombinant insect cells have also been transfected or transformed with nucleic construct(s) for expressing AAV Rep proteins (e.g., Rep AAV2 proteins).

In some embodiments, the recombinant insect cells have further been transfected or transformed with a transgene-containing nucleic acid construct. According to these embodiments, the transgene-containing nucleic acid construct can be comprised in a vector functional in insect cells, such as a baculovirus vector.

For baculovirus vectors and baculovirus DNA, as well as insect cell culture procedures, see, for example, O'Reilly et al., Baculovirus Expression Vectors: A Laboratory Manual, Oxford University Press, New York, 1994, incorporated herein by reference in its entirety. A baculovirus vector that can be used in the context of the present disclosure may contain specific elements, such as an origin of replication, one or more selectable markers allowing amplification in the alternative hosts, such as E. coli and yeast.

Baculoviruses are commonly used for the infection of insect cells for the expression of recombinant proteins. In particular, expression of heterologous genes in insects can be accomplished as described in for instance U.S. Pat. No. 4,745,051; Friesen et al (1986); EP 127,839; EP 155,476; Vlak et al (1988); Miller et al (1988); Carbonell et al (1988); Maeda et al (1985); Lebacq-Verheyden et al (1988); Smith et al (1985); Miyajima et al (1987); and Martin et al (1988), Numerous baculovirus strains and variants and corresponding permissive insect host cells that can be used for protein production are described in Luckow et al (1988), Miller et al (1986); Maeda et al (1985) and McKenna (1989).

Insect host cells include, for example, Lepidopteran cells, and particularly preferred are Spodoptera frugiperda, Bombyx mori, Heliothis virescens, Heliothis zea, Mamestra brassicas, Estigmene acrea or Trichoplusia insect cells. Non-limiting examples of insect cell lines include, for example, Sf21, Sf9, High Five (BT1-TN-5B1-4), BT1-Ea88, Tn-368; mb0507, Tn mg-1, and Tn Ap2, among others.

The Sf9 cells can be cultured under the conditions generally known to a skilled artisan (see, J. Gen. Virol, 36, 361-364 (1977)). Suitable culture conditions can easily be determined by preliminary experiment but, it is preferred to culture in a serum free medium at 27-28° C. Methods of recovering the expressed protein from the cells are not particularly limited and can use, for example, biochemical purification (e.g., affinity chromatography using antibodies to, Japanese encephalitis virus).

Methods for Producing AAV Particles in Insect Cells

In another aspect, this disclosure relates to methods for producing AAV particles, and especially for producing recombinant AAV particles in insect cells. In some embodiments, the method comprises the steps of: (a) culturing an insect host cell as described herein under conditions such that AAV particles are produced; and, (b) collecting the AAV particles that are produced at step (a).

Thus, the AAV particles can be recombinant AAV particles such as those described in the present specification for the purpose of being subsequently used in gene therapy methods.

Growing conditions for insect cells in culture, and production of heterologous products in insect cells in culture, are well-known in the art.

In some embodiments, the method for producing AAV particles defined above further comprises a step of purification of the AAV particles that are collected at step b).

A number of methods for purifying AAV particles, and especially for purifying recombinant AAV particles, are known to one skilled in the art.

As disclosed in the Examples herein, the inventors have found that purification of the AAV-particles can be efficiently performed using an immunoaffinity chromatography purification step.

In some embodiments, the affinity chromatography purification step is performed using an immunoaffinity chromatography support that allows for the purification of AAV8 particles.

The term “immunoaffinity chromatography” as used herein designates any method that uses immobilized antibodies, or fragments thereof, in affinity chromatography.

The term “antibodies or binding fragments thereof” includes monoclonal and polyclonal antibodies, naturally and non-naturally occurring antibodies, whole antibodies and fragments thereof, including fragment antigen-binding such as Fv, Fab and F(ab′)₂ regions, complementarity determining regions (CDRs), single-domain antibodies, nanobodies, and mixtures thereof.

The term “binding fragment thereof” may encompass any fragment of an antibody that can be obtained by deleting part of the original antibody, including, in a non-limiting manner, any antibody of which the Fc region or parts of the variable region (including CDRs) have been deleted.

When immobilized onto the chromatography support, the term encompasses any of the aforementioned variants as long as it retains its ability to bind to at least one epitope at the surface of the rAAV particles to be purified.

In particular, such antibodies or fragments thereof may include isotypes of the IgA, IgD, IgE, IgG and IgM subclasses. According to some embodiments, the antibodies or fragments thereof are monoclonal. Antibodies may be naturally-occurring or non-naturally occurring. They may be of human and/or non-human origin. According to some embodiments, the antibodies are single-chain antibodies, such as the ones obtained by immunization of camelids including dromedaries, camels, llamas, and alpacas; or sharks.

In some embodiments, the immunoaffinity chromatography support is a support onto which an anti-AAV8 antibody or an AAV8-binding fragment thereof is immobilized.

A binding fragment of an anti-AAV8 antibody encompasses molecules, and especially proteins, comprising three Complementary Determining Regions (CDRs) or more from an anti-AAV8 antibody. Binding fragments of an anti-AAV8 antibody encompass Fab, F(ab′)2, a single domain antibody, a ScFv, a Sc(Fv)₂, a diabody, a triabody, a tetrabody, an unibody, a minibody and a maxibody.

Numerous anti-AAV8 antibodies are available to the one skilled in the art. Illustratively, anti-AAV8 monclonal antibodies may be selected from: the anti-AAV8 clone ADK8 commercialized by LSBio under the reference no LS-0200921 or commercialized by MyBioSOurce under the reference no MBS833332, or the anti-AAV8 antibodies described by Tseng et al. (2016, J Virol Methods, Vol. 236: 105-110).

In some embodiments, the affinity chromatography support may be cross-linked poly(styrene-divinylbenzene) onto which the anti-AAV8 antibody or the AAV8-binding fragment thereof is immobilized. In some embodiments, the affinity chromatography support consists of microparticles of poly(styrene-divinylbenzene) on which an anti-AAV8 antibody or an AAV8-binding fragment thereof is immobilized.

Illustratively, it may be used the chromatography support commercialized under the name of POROS™ CaptureSelect™ AAV8 under the reference no A30793 by Thermo Fischer Scientific (Waltham, Mass., USA). POROS™ CaptureSelect™ AAV8 resins are 50 μm, rigid, polymeric affinity chromatography resins designed for the purification of adeno-associated virus subtype 8. This resin backbone consists of crosslinked poly[styrene divinylbenzene] and is coated with a cross-linked polyhydroxylated polymer. This coating is further derivatized with an affinity ligand which is a single-domain [V_{H}H] monospecific anti-AAV8 antibody fragment.

Thus, according to another aspect, the present disclosure relates to a method for purifying AAV (e.g., AAV-Anc80L65) particles, comprising a step of affinity chromatography with a support onto which an anti-AAV8 antibody or an AAV8-binding fragment thereof is immobilized.

In some embodiments, the affinity chromatography support is cross-linked poly(styrene-divinylbenzene) on which an anti-AAV8 antibody or an AAV8-binding fragment thereof is immobilized. In some embodiments, the affinity chromatography support consists of microparticles of poly(styrene-divinylbenzene) on which an anti-AAV8 antibody or an AAV8-binding fragment thereof is immobilized.

Illustratively, the chromatography support commercialized under the name of POROS™ CaptureSelect™ AAV8 under the reference no A30793 by Thermo Fischer Scientific (Waltham, Mass., USA) may be used.

In some embodiments of the purification method, the affinity chromatography step is the sole separation step. Thus, in some embodiments, the purification method does not comprise further steps of chromatography, irrespective of the kind of chromatography is concerned (e.g. size exclusion chromatography, non-AAV8 affinity chromatography supports, anion exchange chromatography, cation exchange chromatography, etc.).

In some embodiments, the affinity chromatography step may be followed by one or more additional separation steps, such as ion exchange chromatography step(s), which encompass anion chromatography step(s) and/or cation chromatography step(s).

Additional separation steps may be performed notably for discarding the empty capsid particles, as is conventional in a number of known methods for purifying recombinant AAV particles.

Thus, this disclosure also relates to a method for purifying AAV particles comprising the steps of: a) providing a sample comprising AAV particles, b) subjecting the sample provided at step a) to a step of imunoaffinity chromatography with a chromatography support onto which an anti-AAV8 antibody or an AAV8-binding fragment thereof is immobilized, c) collecting the purified AAV8 particles obtained at the end of step b).

In some embodiments, step a) comprises the steps of: a1) disrupting the cells contained in a sample of cultured recombinant cells producing AAV particles, whereby a AAV-containing lysate sample is provided, and a2) subjecting the AAV-continuing sample provided at step a1) to a depth filtration, whereby an enriched AAV-containing sample is provided. Thus, in some embodiments of step a) of the purification method, the sample comprising AAV particles may consist of a AAV-containing cell lysate.

Also, in some embodiments of step a) of the purification method, the sample comprising AAV particles may consist of a cell lysate that has been enriched in AAV particles by being subjected to a step of depth filtration, as in the embodiments of the method comprising steps a1) and a2).

In some embodiments, step a1) comprises the steps of: a1.1) disrupting the cells contained in a sample of cultured recombinant cells producing AAV particles, whereby a AAV-containing lysate sample is provided, and a1.2) clarifying the AAV-containing lysate sample provided at step a1.1) by mixing the said sample with an endonuclease composition, whereby a clarified AAV-containing lysate composition is provided.

As used herein, the term “lysate”, in relationship with a purification method of AAV particles, encompasses both an unclarified lysate and a clarified lysate. Notably, the AAV-containing lysate sample which is provided at step a1) of the purification method (i) may consist of an unclarified AAV-containing lysate sample or (ii) may consist of a clarified AAV-containing lysate composition such as that which is provided at step a.1.2.) of the corresponding embodiments of the purification method.

As it is readily understood by the one skilled in the art, the sample which is provided at step a) of the purification method may be selected from a group comprising (i) an unclarified lysate or (ii) a clarified lysate, such as that which is provided at the end of step a.1.2.) in some embodiments of the purification method.

As is readily understood by one skilled in the art, the clarified AAV-containing sample provided at the end of step a2) consists of the sample provided at step a) which is subjected to a step of immunoaffinity chromatography at step b) of the purification method.

In some embodiments, the purification method further comprises a step d) of subjecting the AAV particles collected at step c) to a tangential flow filtration.

In some embodiments, the purification method further comprises a step e) of sterilization of the AAV particles obtained at the end of step c).

It has been shown in the examples that an optimal purification of the AAV particles by performing the purification method described herein may be reached when the purification method is performed in optimal conditions.

Thus, in some embodiments of the conditions of step b) of immunoaffinity chromatography, the AAV particles bound to the immunochromatography support are eluted in strong acidic conditions (e.g., at a pH below 3.0).

Various steps of the purification methods described herein are described in more detail below.

Depth Filtration

Depth filtration allows one to discard a major part of contaminant DNA and proteins. This step renders possible the purification of rAAV particles through immunoaffinity chromatography directly from a rAAV-containing composition, and especially from a rAAV-containing clarified composition.

According to some embodiments, the starting material used at step a) is a cell lysate obtained by contacting a culture of cells, which encompass a culture of insect cells producing rAAV particles, with a composition comprising at least a detergent or a surfactant so that the cells are disrupted, so as to provide an unclarified AAV-containing lysate composition.

Examples of suitable detergents for cell lysis include Triton X-100, Triton X-114, NP-40, Brij-35, Brij-58, Tween 20, Tween 80, Octyl glucoside, Octyl thioglucoside, SDS, CHAPS and CHAPSO.

According to some embodiments, the unclarified AAV-containing lysate composition used at step a) is brought into contact with a composition comprising a nuclease such as a DNAse and/or a RNAse, so as to obtain a clarified AAV-containing composition. As a nuclease, one skilled in the art may use a genetically engineered endonuclease from Serratia marcesens that degrades all forms of DNA and RNA (single-stranded, double-stranded, linear and circular), such as the nuclease marketed under the name Benzonase® Nuclease by Sigma Aldrich.

The clarification step described above may be performed according to the manufacturer's recommendations. Illustratively, the step of clarification using a nuclease such as Benzonase® may be performed at 37° C. during a period of time ranging from 1.5 h to 3.0 h (e.g., a time period of about 2.5 h).

At step a1) of depth filtration, any depth filter membrane known to those skilled in the art may be used. According to some embodiments, step a1) of depth filtration is performed using a depth filter membrane comprising a layer of borosilicate glass microfibers and a layer of mixed esters of cellulose. According to one exemplary embodiment, step a1) is performed using a Polysep™ II (Millipore®) filter.

Immunoffinity Chromatography

In some embodiments, step b) is performed using an antibody that binds specifically to at least one epitope that is present on the AAV particles. As is shown in the Examples herein, an antibody that binds specifically to at least one epitope that is present on the AAV particles encompasses an antibody directed to an AAV8, as well as an AAV8-binding fragment thereof.

Anti-AAV8 antibodies and AAV8-binding fragments thereof may be obtained and immobilized onto supports using a variety of techniques that range from covalent attachment to adsorption-based methods, as described, for instance, in Moser & Hage (“Immunoaffinity chromatography: an introduction to applications and recent developments”; Bioanalysis; 2(4): 769-790; 2010).

Anti-AAV8 monoclonal antibodies may be prepared using any technique which provides for the production of antibody molecules, e.g., by continuous cell lines in culture. These include, but are not limited to, the hybridoma technique, the human B-cell hybridoma technique, and the EBV-hybridoma technique (Kohler et al., 1975, Nature 256:495-497; Kozbor et at, 1985, J. Immunol. Methods 81:31-42; Cote et al., 1983, Proc. Natl. Acad. Sci. 80:2026-2030; Cole et al, 1984, MoL Cell Biol. 62:109-120).

A number of suitable immunoaffinity chromatography supports for use with the present methods are known and include without limitation, Affi-Gel (Biorad); Affinica Agarose/Polymeric Supports (Schleicher and Schuell); AvidGel (BioProbe); Bio-Gel (BioRad); Fractogel (EM Separations); HEMA-AFC (Alltech); Reacti-Gel (Pierce); Sephacryl (Pharmacia); Sepharose (Pharmacia); Superose (Pharmacia); Trisacryl (IBF); TSK Gel Toyopearl (TosoHaas); Ultragel (IBF); AvidGel CPG (BioProbe); HiPAC (ChromatoChem); Protein-Pak Affinity Packing (Waters); Ultraffinity-EP (Bodman) and Emphaze (3M Corp./Pierce).

Other chromatography supports include affinity monolith chromatography supports, and POROS® affinity chromatography supports.

In some embodiments, step b) of the purification method is performed using a chromatography support consisting of POROS™ CaptureSelect™ AAV8 under the reference no A30793 by Thermo Fischer Scientific (Waltham, Mass., USA).

According to some embodiments, the rAAV-containing clarified composition is loaded on a immunoaffinity chromatography column that has previously been pre-equilibrated with a PBS 1× equilibration buffer at pH 7.5.

In some embodiments, the immunoaffinity column is pre-equilibrated with a volume of equilibration buffer, e.g., five times the volume of the immunoaffinity support. In some embodiments, the pre-equilibration buffer may be a PBS 1× buffer, such as the PBS 1× buffer commercialized by Lonza under the reference number BE17-516F. In some embodiments, pre-equilibration is performed at a pH of 7.5.

According to some embodiments, the pH of the rAAV-containing clarified composition is at a neutral to basic pH (e.g., a pH ranging from 6.0 to 8.0), prior to loading on the immunoaffinity column.

In some embodiments of the conditions of step b) of immunoaffinity chromatography, the AAV particles bound to the immunochromatography support are eluted in strong acidic conditions (e.g., at a pH below 3.0). In some embodiments of step b) of the AAV purification method, the elution step is performed at a pH below 3.0 (e.g., a pH ranging from 1.5 to 3.0). In some embodiments of step b) of the AAV purification method, the elution step is performed at a pH ranging from 2.5 to 1.5; which encompasses a pH ranging from 2.3 to 1.7, which includes a pH ranging from 2.2 to 1.8, which pH may range from 2.1 to 1.9.

In some embodiments of step b), the elution step is performed using a buffer such as a PBS buffer at the strong acidic pH conditions specified above. Illustratively, a PBS buffer comprising 137 mM NaCl, 2.7 mM KCl, 10 mM NaH₂PO₄ and 1.76 mM KH₂PO₄ may be used. Once eluted, the pH of the rAAV-enriched composition can be neutralized in a manner suitable for obtaining a rAAV-enriched composition with a neutral or basic pH, which includes a pH of 8.0 or above (e.g., a pH of 8.5). The reason is that rAAV particles tend to lose their integrity and/or infectivity if maintained in a composition having an acidic pH.

In some embodiments, the eluted fraction(s) containing the AAV particles are neutralized. Illustratively, neutralization may be performed by adding 0.1 volume of a Tris-HCl buffer at pH 8.0 to 1 volume of an eluted fraction. In some embodiments, the first rAAV enriched composition can be supplemented with a non-ionic surfactant (e.g., Pluronic® F-68 (Gibco)) before, during, or after neutralization. A non-ionic surfactant can be present in an amount ranging from 0.0001% to 0.1% (v/v) of the total volume of the composition (e.g., an amount ranging from 0.0005% to 0.005% (v/v) of the total volume of the composition; e.g., about 0.001% (v/v) of the total volume of the composition).

The use of a non-ionic surfactant, as defined above, and in the other steps, further contributes to the efficiency and scalability of the method. In particular, the use of a non-ionic surfactant, as defined above, prevents the aggregation or adherence of rAAV particles, before, during and after purification.

Tangential Flow Filtration and Subsequent Steps

Tangential Flow Filtration (TFF) is a polishing step, which allows one to discard small-sized particle-related impurities through cycles of concentration and diafiltration through the pores of the filter. This polishing step has the other advantage of being suitable for changing the buffer of the eluted fractions and for concentrating the rAAVs. TFF, e.g., Alternating Tangential Flow (ATF) filtration, can be achieved using, for example, a hollow fiber filter.

According to one embodiment, tangential flow filtration at step b) is performed by using a filter membrane having a molecular weight cut-off value equal or inferior to 150 kDa (e.g., ranging from 20 kDa to 150 kDa, 25 kDa to 150 kDa, or about 100 kDa). According to some embodiments, salts and/or detergents and/or surfactants and/or nucleases can added during, before or after the TFF or ATF.

According to some embodiments, the method may further include a step of treatment with detergents, surfactants, and/or nucleases, including DNAses, during, before or after the TFF.

In some embodiments, the AAV particles are diafiltered and concentrated in the presence of a non-ionic surfactant (e.g., Pluronic® F-68 (Gibco)). The non-ionic surfactant can be present in an amount ranging from 0.0001% to 0.1% (v/v) of the total volume of the composition (e.g., an amount ranging from 0.0005% to 0.005% (v/v) of the total volume of the composition; e.g., about 0.001% (v/v) of the total volume of the composition).

Also advantageously, the AAV particle-containing composition can be diafiltered and concentrated against a Saline Ocular Solution, dPBS, dPBS+Mg/Ca or Ringer's Lactate, which may further comprise a non-ionic surfactant as defined above.

According to some embodiments, the purified recombinant AAV particles obtained at step b) are sterilized. For example, the purified recombinant AAV particles can be submitted to a step of sterile filtration over a filter membrane having a pore size of 0.30 μm or less (e.g., 0.25 μm or less). For example, a filter membrane having a pore size of 0.22 μm can be used.

The disclosure also relates to purified rAAV particles obtained by performing a method as described above.

Characterization of the Purified rAAV Particles

Advantageously, the above-mentioned methods can be used for obtaining purified recombinant AAV particles that are suitable for gene therapy and/or for preparing a medicament for gene therapy.

The purity of recombinant AAV particle preparations also has important implications for both safety and efficacy of clinical gene transfer. The methods used to purify AAV particles can dramatically influence the purity of the preparation in terms of residual host cell proteins and/or baculovirus proteins. The purity of the preparation can be assessed by sodium dodecyl sulfate-polyacrylamide gel electrophoresis (SDS-PAGE) and Coomassie Blue or silver stained.

Vector particle concentration can be assessed by quantitative PCR (of, e.g., genome containing particles) as shown in the Examples herein.

VP1, VP2, and VP3 can be encoded by nucleic acids comprised in the nucleic acid sequences of SEQ ID NO:3 or 4.

The above-mentioned sequences are given as reference sequences.

Thus, the term “purity” refers to the absence of general impurities. Purity is expressed as a percentage, and relates to the total amount of VP1, VP2 or VP3 proteins, in comparison to the total amount of detected proteins in a Coomassie Blue or silver-stained polyacrylamide gel.

The term “general impurities” refers to impurities which were present in the starting material but which are not considered as particle-related impurities. Thus, general impurities encompass impurities which are derived from the host cells or baculoviruses but which are not AAV particles.

A “dose” is defined as the volume of preparation that corresponds to a target amount of vector genome (vg) and has been tested to produce a therapeutic effect in preclinical studies. As an example, a dose could be 1 ml of a solution containing 1×10¹³ vg/ml.

Infectious particle concentration can be assessed by transfecting reporter cells and measuring green forming units (GFU) using a protocol which is well known in the art.

Therapeutic Methods

As it is already described elsewhere in the present specification, embodiments of AAV particles (e.g., AAV-Anc80L65 particles) obtained according to the disclosure consist of recombinant AAV particles comprising one or more transgene nucleic acid constructs of interest encapsidated therein, which recombinant AAV particles can be used in therapeutic methods, e.g., methods of gene therapy.

According to aspects of the present disclosure, purified recombinant AAV particles obtained according to the present disclosure may be used for therapeutic treatment of conditions or diseases, especially according to methods of gene therapy.

The recombinant AAV particles obtained according to the methods described herein may be delivered to a subject in compositions according to any appropriate methods known in the art. The rAAV, for example, suspended in a physiologically compatible carrier (e.g., in a composition), may be administered to a subject, e.g., host animal, such as a human, mouse, rat, cat, dog, sheep, rabbit, horse, cow, goat, pig, guinea pig, hamster, chicken, turkey, or a non-human primate (e.g, chimpanzee or macaque). In some embodiments, a host animal does not include a human.

Delivery of the rAAVs to a mammalian subject may be by, for example, intramuscular injection or by administration into the bloodstream of the mammalian subject. Administration into the bloodstream may be by injection into a vein, an artery, or any other vascular conduit. In some embodiments, the rAAVs are administered into the bloodstream by way of isolated limb perfusion, a technique well known in the surgical arts, the method essentially enabling the artisan to isolate a limb from the systemic circulation prior to administration of the rAAV virions.

Such pharmaceutical compositions may comprise recombinant AAV particles alone, or in combination with one or more other virus-derived particles (e.g., a second rAAV encoding having one or more different transgenes). In some embodiments, a composition comprises 1, 2, 3, 4, 5, 6, 7, 8, 9, 10, or more different rAAVs each having one or more different transgenes.

Suitable carriers may be readily selected by one of skill in the art in view of the indication for which the rAAV is directed. For example, one suitable carrier includes saline, which may be formulated with a variety of buffering solutions (e.g. phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

The dose of recombinant AAV particles required to achieve a particular “therapeutic effect,” e.g., the units of dose in genome copies/per kilogram of body weight (GC/kg), will vary based on several factors including, but not limited to: the route of administration, the level of transgene expression required to achieve a therapeutic effect, the specific disease or disorder being treated, and the stability of the transgene nucleic acid or polypeptide product. One of skill in the art can readily determine a rAAV dose range to treat a patient having a particular disease or disorder based on the aforementioned factors, as well as other factors that are well known in the art.

An effective amount of a recombinant AAV is an amount sufficient to target infect an animal, target a desired tissue. In some embodiments, an effective amount of a recombinant AAV is an amount sufficient to produce a stable somatic transgenic animal model. The effective amount will depend primarily on factors such as the species, age, weight, health of the subject, and the tissue to be targeted, and may thus vary among animal and tissue. For example, an effective amount of the rAAV is generally in the range of from about 1 ml to about 100 ml of solution containing from about 10⁹ to 10¹⁵ genome copies/mL. In some embodiments, the rAAV is administered at a dose of 10¹⁰, 10¹¹, 10¹², 10¹³, 10¹⁴, or 10¹⁵ genome copies per subject. In some embodiments, the rAAV is administered at a dose of 10¹⁰, 10¹¹, 10¹², 10¹³, or 10¹⁴genome copies per kg.

### Examples

The present invention is further illustrated, without in any way being limited to, the Materials and Methods and Examples below.

Materials and Methods

Characterization of Recombinant Baculovirus

The identity of the baculoviral genomes was verified by Sanger sequencing from PCR products of P2 stock DNA extracts. The infectious titer of BEV (Baculovirus Expression Vector) stocks was determined by Cell Size Assay (CSA) (Janakiraman et al., 2006, J. Virol. Methods, 132 (1-2):48-58).

Recombinant AAV Production

For rAAV production in insect cells, Spodoptera frugiperda Sf9 cells were grown at 27° C. in Sf-900 III SFM in a spinner flask or 2-L bioreactor cultures (Thermo Fisher Scientific, USA). Sf9 cells were infected at a density of 10⁶ cells per mL with a BEV-rep/cap and a BEV-AAV-GFP at an MOI of 1 (CSA) per baculovirus.

Characterization of AAV Vectors

To detect viral proteins in cell cultures by Western blot, 10 μg of total proteins were extracted in RIPA buffer from Sf9 extracts. To detect VP proteins in purified rAAV stocks, 1×10¹¹ vector genomes were diluted up to 20 μL with sterile water. After addition of 5 μL of Laemmli buffer (5×), samples were boiled for 5 min at 95° C. and loaded on 8 Novex® 10% tris-glycine polyacrylamide gels (Thermo Fisher Scientific). Subsequently, proteins were transferred to a nitrocellulose membrane (Life Sciences, Biorad, Calif., USA) through semi-dry blotting and blocked with 1×PBS, 1% Tween-20 and 5% milk overnight at 4° C. Primary monoclonal B1 (Cat. 61058, Progen Biotechnik) and polyclonal anti-VP antibodies (Cat. 61084, Progen Biotechnik) were used at 1:10 and 1:500 dilution, respectively, to detect AAV capsid proteins VP1, VP2 and VP3. Anti-mouse 303.9 antibody (Cat. 65169, Progen Biotechnik) was diluted at 1:20 in blocking buffer for Rep proteins recognition. The following Horseradish peroxidase-conjugated secondary antibodies were used for detection of primary signal: goat anti-mouse antibody at 1:2000 dilution (P0447, Dako) or rabbit anti-goat antibody at 1:2000 dilution (P0449, Dako). Western blotting Pierce™ ECL substrate (ThermoFisher Scientific) was used to visualize bound antibodies.

Sf9 cells were infected by BEV and harvested at different time points after-infection. Western blot analysis of cells revealed the expression of AAP using both expression cassettes (OptMin and IntronMin) for the Anc80L65 serotype. Non-infected cells were used as negative controls and a BEV expressing Rep2Cap2 was used as a positive control.

For qPCR analysis, 3 μL of each purified rAAV stock was pretreated or not with 20 U of DNase I (Roche, Bale, Switzerland) before DNA extraction in a total volume of 200 μL of DNase reaction buffer (13 mM Tris pH 7.5, 0.12 mM CaCl₂, 5 mM MgCl₂) for 45 min at 37° C. The vector genome (vg) copy number was determined after DNA extraction using the High Pure Viral Nucleic Acid kit (Roche, Bale, Switzerland) by free ITR assays.

Vector purity was evaluated by SDS-PAGE followed by silver staining (PlusOne™ Silver Stain kit, GE Healthcare, Little Chalfont, UK) of 2×10¹⁰ vector genomes of each rAAV stock. The vector genome (vg) copy number was determined by free ITR qPCR assay (D'Costa et al., 2016, Mol Ther Methods Clin Dev, 30 (no 5):16019-doi: 10.1038/mtm.2016.19), HBB2 pA qPCR assay using the primers 5′-AGG TGA GGC TGC AAA CAG CTA (SEQ ID NO:5), 5′-TTT CTG AGG GAT GAA TAA GGC ATA G (SEQ ID NO:6) and probe 5′-FAM-TGC ACA TTG GCA ACA GCC CCT GAT G-TAMRA (SEQ ID NO:7) or the eGFP qPCR assay using the primers 5′-AGT CCG CCC TGA GCA AAG A (SEQ ID NO:8), 5′-GCG GTC ACG AAC TCC AGC (SEQ ID NO:9) and the probe 5′-FAM-CAA CGA GAA GCG CGA TCA CAT GGT C-TAMRA (SEQ ID NO:10).

Baculoviral DNA contamination was quantified by Bac (AcMNPV DNA polymerase) qPCR using the primers 5′-ATT AGC GTG GCG TGC TTT TAC (SEQ ID NO:11), 5′-GGG TCA GGC TCC TCT TTG C (SEQ ID NO:12) and probe 5′-FAM-CAA ACA CGC GCA TTA ACG AGA GCA CC-TAMRA (SEQ ID NO:13). The copy number of the rep and cap sequences was determined using the following sets of primers and probe Rep52F 5′-GCC GAG GAC TTG CAT TTC TG (SEQ ID NO:14), Rep52R 5′-TCG GCC AAA GCC ATT CTC (SEQ ID NO:15), Rep52P 5′-FAM-TCC ACG CGC ACC TTG CTT CCT C-TAMRA (SEQ ID NO:16) for rep and Cap8F 5′-TTC TGC AGC TCC CAT TCA ATT (SEQ ID NO:17), Cap8R 5′-TCA ACC ACTT CAA AGC TGA ACT CTT (SEQ ID NO:18) Cap8P 5′-FAM-CCA CGC TGA CCT GTC CGG TGC-TAMRA (SEQ ID NO:19) for cap8Infectivity of AAV vectors were tested in HeLa cells seeded in 24-well plates and infected with AAV vectors at different multiplicity of infection in triplicates. Cells were observed 48 hours post-infection, Green Forming Units were counted in serial dilutions and the infectivity was expressed as GFU/mL.

### Example 1: Construction of the AAV-Anc80L65 Vectors

1.1. Plasmid Cloning

Two DNA fragments, named P10-CapAnc80L65start_OPT (SEQ ID NO. 30) and P10-CapAnc80L65start_IntronP10 (SEQ ID NO:31), were synthesized (Genewiz (NJ, USA)) and cloned in pUC57-Kan plasmid. In SEQ ID NOs: 1 and 2 shown below, BstZ17I, BsiWI and NsiI enzymatic sites used for further cloning are underlined in italic letters, and mutations in the Anc80L65-L0065 cap coding sequence (CDS) are indicated by bold underlined letters. In SEQ ID NO:2, the intron-P10 sequence is highlighted in grey.

1.1.1. OptMin Construct

The donor plasmid 664_pSR Rep2CapAnc80L65_Opt (illustrated in FIG. 7) contains the ancestral cap CDS Anc80L65-L0065 optimized for the expression in Sf9 insect cell line (CapAnc80L65_Opt), under the transcriptional control of the baculoviral p10 promoter and followed by the herpes simplex virus type 1 thymidine kinase polyadenylation signal (HSVtk-pA), and the AAV-2 rep CDS optimized as described by Smith et al. 2009 for the expression of rep78/52 proteins in insect cells, under the control of the baculoviral polyhedrin (polh) promoter and followed by the simian virus 40 late polyadenylation signal (SV40-pA).

The CapAnc80L65 sequence was optimized based on the assumption that mutating the AUG start codon of VP1 in ACG allows for some 40S ribosomal subunits to bypass the initial codon and begin translation at further downstream start codon (ribosome leaky scanning mechanism). Thus, the ATG start codon of VP1 was mutated in ACG (T to C at position 2 of cap CDS, M to T) and an additional out-frame ATG before VP2 start codon was changed in ACG (T in C at position 12 of cap CDS, silent mutation). Since CUG triplet corresponds to the start codon of AAP for AAV serotypes 1 through 13 (Sonntag et al. 2001), the putative start codon of the assembly-activating protein (AAP) of Anc80L65-L0065 was also mutated from CAG to CTG (at position 528 of cap CDS, Q to L in AAP, silent for VP1/2 proteins).

The donor plasmid 664 was generated as follows: (1) the BstZ17I-NsiI fragment of the P10-CapAnc80L65start_OPT synthetic sequence (SEQ ID NO: 1) was ligated with the BstZ17I-NsiI fragment of the pSR660_Rep2Cap8 plasmid, replacing the beginning of cap8 sequence by capAn80 optimized sequence, and (2) the BsiWI-SpeI fragment of the plasmid 549_pAAVvector2Anc80L65-L0065 Trimmed was inserted in the plasmid generated at step 1 between BsiWI and NheI restriction sites to assemble the full-length CapAnc80L65_Opt CDS. The nucleic acid sequence of the OptMin-containing donor plasmid can be found in SEQ ID NO. 26.

1.1.2. IntronMin Construct

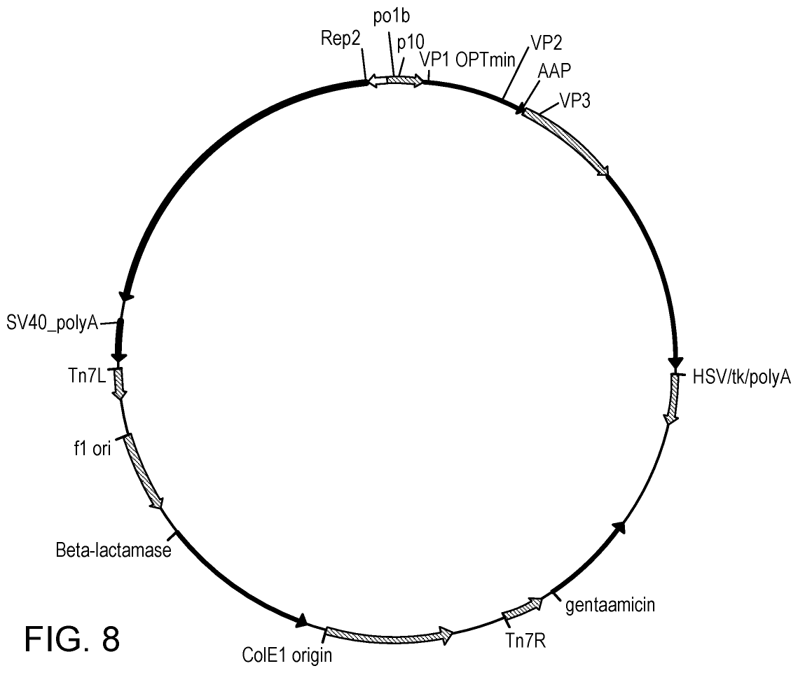

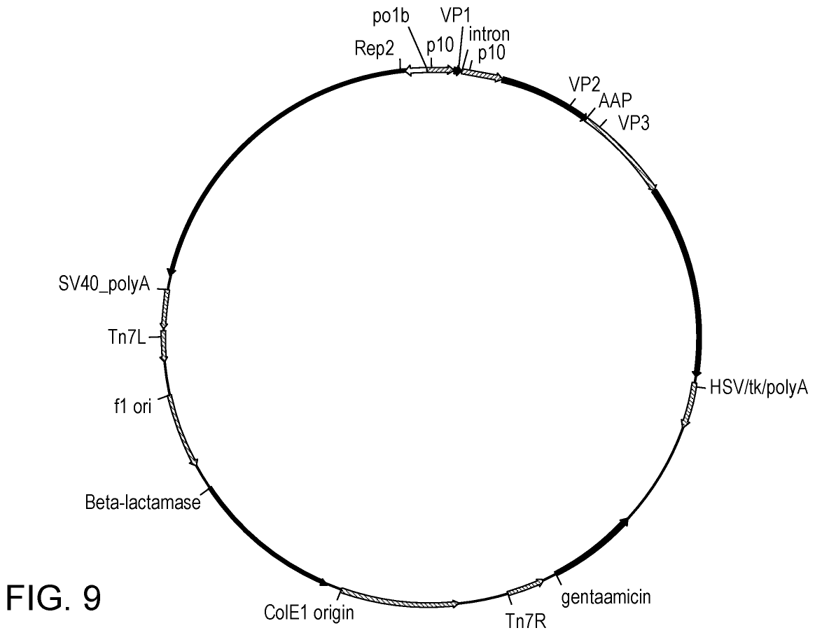

The donor plasmid 665 Rep2CapAnc80L65 IntronMin (Illustrated in FIG. 8) contains the ancestral cap CDS Anc80L65-L0065 with an internal synthetic intron described below (CapAnc80L65 IntronP10), under the transcriptional control of the baculoviral p10 promoter and followed by the herpes simplex virus type 1 thymidine kinase polyadenylation signal (HSVtk-pA), and the AAV-2 rep CDS as described above.

In the P10-CapAnc80L65start_IntronP10 synthetic sequence (SEQ ID NO:2), the synthetic intron corresponds to the intron described by H. Chen in (Chen, 2008) but in our design the polyhedrin promoter was replaced by the p10 promoter at the same position. The intron-p10 was inserted in the CapAnc80L65 gene between nucleotide 25 and 26 of cap CDS, similarly to the location described by H. Chen in the AAV-2 cap CDS (Chen, 2008).

The out-frame ATG at position 12 of the cap CDS was changed to ACG as described above. Furthermore, the AAP start codon of Anc80L65-L0065 was mutated from CAG to CTG (at position 782 of CapAnc80L65-intron sequence, Q to L in AAP, silent for VP1/2 proteins).

The donor plasmid 665_pSR Rep2CapAnc80L65_Intron was generated as follows: (1) the BstZ17I-NsiI fragment of the P10-CapAnc80L65start_IntronP10 synthetic sequence (SEQ ID NO: 2) was ligated with the BstZ17I-NsiI fragment of the pSR660_Rep2Cap8 plasmid replacing the beginning of cap8 sequence by capAn80-intronp10 sequence and (2) the BsiWI-SpeI fragment of the plasmid 549_pAAVvector2Anc80L65-L0065 Trimmed was inserted in the plasmid generated at step 1 between BsiWI and NheI restriction sites. The nucleic acid sequence of the IntronMin-containing donor plasmid can be found in SEQ ID NO. 27.

1.1.3. Transgene Constructs

Plasmid pMB-eGFP-Puro is derived from the pFastBac™ Dual plasmid (Thermo Fisher Scientific) and contains a human cytomegalovirus (CMV) promoter, the enhanced green fluorescent protein (eGFP) reporter gene, followed by an EMCV internal ribosome entry site (IRES), a puromycin resistance sequence, and the 3′ untranslated region (3′-UTR) of the human hemoglobin beta (HBB) gene. The recombinant AAV genome in pMB-eGFP-Puro is delimited by the wild-type flip and flop ITRs from AAV serotype 2.

The pFB-eGFP plasmid is identical to the pMB-eGFP-Puro plasmid but lacks the IRES and the puromycin cDNA and contains truncated ITRs of AAV-2 derived from plasmid pSub-201 (Samulski et al., 1987, J Virol, Vol. 61(10):30963101). The nucleic acid of plasmid pMB-eGFP-Puro is found as SEQ ID NO:28 herein. The nucleic acid of plasmid pFB-eGFP may found as SEQ ID NO:29 herein. The donor plasmids were validated by Sanger sequencing and subsequently used for the generation of the recombinant baculoviruses.

1.2. Generation of Recombinant Baculovirus

The BEV-eGFP and BEV-GFP-Puro carries the ITRs of AAV-2 and the expression cassette of GFP under the expression of ubiquitous promoter. These recombinant baculoviruses were generated using the donor plasmids pFB-GFP and pMB-GFP-Puro, respectively. BEV-rep2capAnc80L65_intron and BEV-rep2capAnc80L65_opt were generated using the donor plasmids 664 and 665 described above.

Tn7 site-specific transposition of the cassette of interest in the bacmid backbone bMON14272 was performed by transformation of 10 ng of the donor plasmids in E. coli DH10Bac™ competent bacteria in accordance with the instructions in the Bac-to-Bac® expression system manual (Thermo Fisher Scientific, USA). The recombinant bacmids were validated for the presence of the insert DNA by PCR using the primers M13-pUC-F 5′-CCA GTC ACG ACG TTG TAA AAC G (SEQ ID NO:20) and M13-pUC-R 5′-AGC GGA TAA CAA TTT CAC ACA GG (SEQ ID NO:21) from either side of the insert and the set of primers M13-pUC-F and BAC-G 5′-AGC CAC CTA CTC CCA ACA TC (SEQ ID NO:22) targeting the gentamycin resistance sequence in the insertion cassette, and by Sanger sequencing.

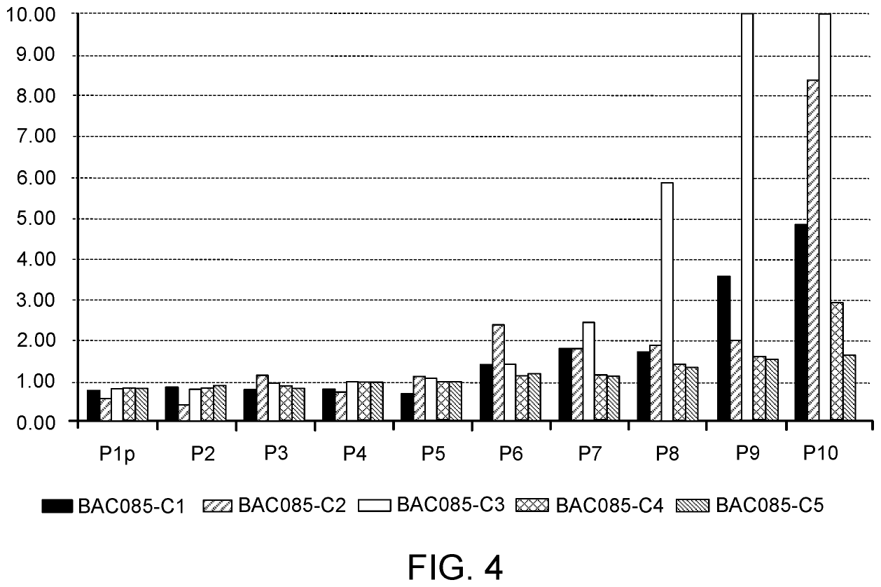

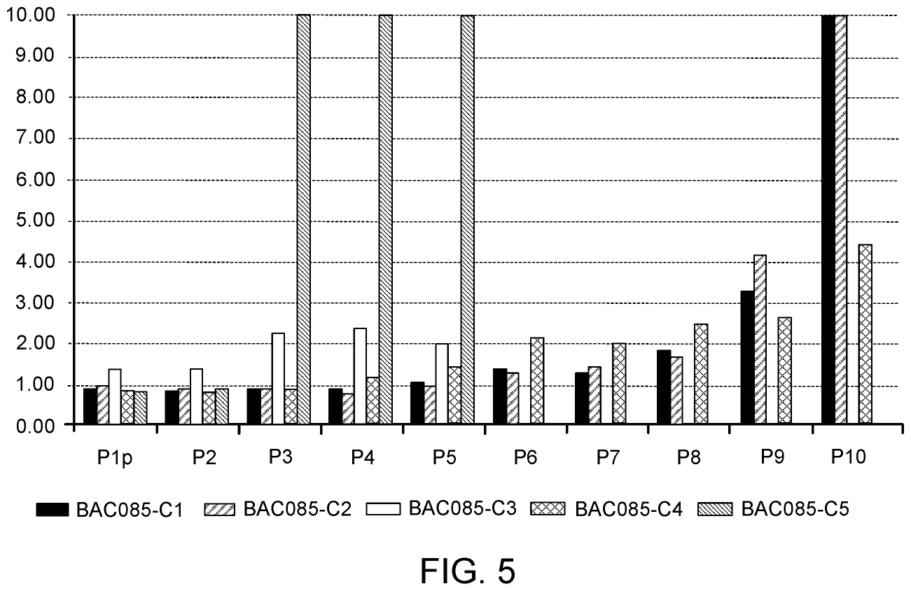

One microgram of each bacmid DNA was then transfected in 10⁶ insect cells cultivated in 6-well plates using 9 μL of Cellfectin® II reagent (ThermoFisher Scientific, USA). The supernatants (P1 stocks) were recovered 96 h post-transfection. Plp clones were then isolated from the P1 stocks by plaque assay. One clone per recombinant baculovirus was selected based on the infectious titer in cell size assay and genetic stability of the insert after five passages. For the BEV genetic stability validation, Sf9 cells were seeded at 1×10⁶ cells per well in a 6-well plate and infected by 2 μL of each Plp supernatant. Three days after infection, cells were harvested and centrifuged for 5 min at 1000×g. Supernatants were recovered and 2 μL (P2) were used for a second round of infection (P3), 2 additional passages were performed using the same methodology up to five infection cycles (P10) (FIGS. 4 and 5).

BEV DNA was extracted from 40 μL of each supernatant using the High Pure Viral Nucleic Acid kit (Roche, Bale, Switzerland) and subjected to a qPCR assay targeted to the ITR of serotype 2 for the BEV-AAV using the primers 5′-GGA ACC CCT AGT GAT GGA GTT (SEQ ID NO:23), 5′-CGG CCT CAG TGA GCG A (SEQ ID NO:24) and probe 5′-FAM-CAC TCC CTC TCT GCG CGC TCG-BHQ (SEQ ID NO:25) or targeted to rep sequence (Rep52 qPCR described below) for BEV-RepCap. The BEV genomic stability was validated if the ratio of the insert copy number (ITR or rep) over the baculoviral DNA polymerase gene copy number (Bac qPCR described below) is stable at least over the 5 passages. The BEV P2 stocks were finally generated after amplification of Plp stocks in S. 19 cells seeded in spinner flasks. P3 stocks were generated from P2 stocks in 2 L glass bioreactor.

### Example 2: Production of Recombinant AAV-Anc80L65 in Insect Cells

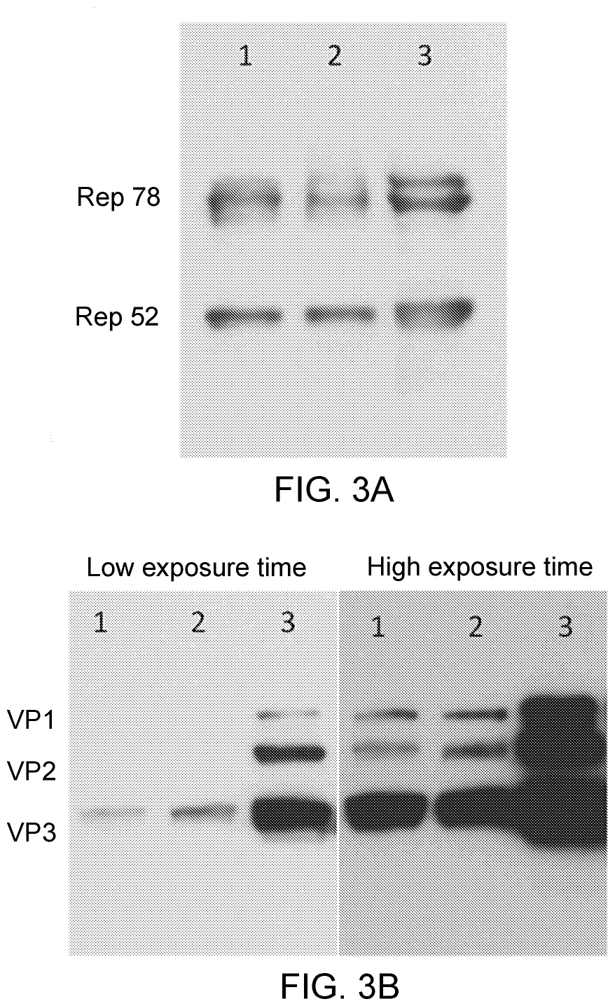

As shown in FIG. 3, Sf9 cells which have been infected with the recombinant baculovirus vectors comprising either the OptMin construct or the IntronMin construct express both the AAV2 Rep proteins (FIG. 3A) and the AAV-Anc80L65 cap proteins (FIG. 3B).

Specifically, in both FIGS. 3A and 3B, Lane 1 contains a sample from Sf9 cells transfected with a baculovirus vector comprising the Anc80L65_OptMin construct (vector Rep2CapAnc80L65_OptMin); Lane 2 contains a sample from Sf9 cells transfected with a baculovirus vector comprising the Anc80L65_IntronMin construct (vector Rep2CapAnc80L65_IntronMin); and Lane 3 contains a sample from Sf9 cells transfected with a baculovirus vector comprising the Rep2Cap8_WT construct encoding the cap proteins of AAV2 (vector Rep2Cap8).

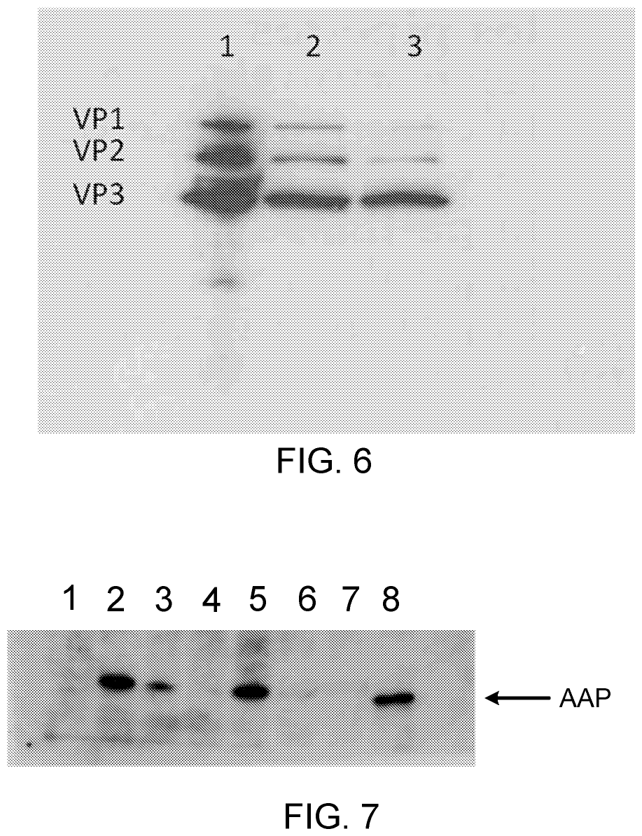

In addition, the results depicted in FIG. 3B and FIG. 6 show that Sf9 cells infected with a baculovirus vector comprising the OptMin construct express each of the AAV-Anc80L65 VP1, VP2 and VP3 proteins, with the VP3 protein being produced predominantly. Specifically, in FIG. 6, Lane 1 contains a sample from Sf9 cells transfected with the control baculovirus vector Rep2cap8 (vector Rep2cap8); Lane 2 contains a sample from Sf9 cells transfected with a baculovirus vector comprising the Anc80L65_OptMin construct from selected clone 3 (passage 2) (vector Rep2CapAnc80L65_OptMin); and Lane 3 contains a sample from Sf9 cells transfected with a baculovirus vector comprising the Anc80L65 IntronMin construct from selected clone 1 (passage 2) (vector Rep2CapAnc80L65 IntronMin). The results depicted in FIGS. 3B and 6 also show that Sf9 cells infected with a baculovirus vector comprising the IntronMin construct express each of the AAV-Anc80L65 VP1, VP2 and VP3 proteins, the VP3 protein being produced predominantly.

Highly importantly, the results depicted in FIG. 7 show that the Anc80_L65 AAP that is encoded in each of the Rep2CapAnc80L65_OptMin and the Rep2CapAnc80L65_IntronMin is actually expressed in the infected cells. Specifically, in FIG. 7, Lane 1 contains a sample of Rep2CapAnc80L65_OptMin, 24 hours post-infection; Lane 2 contains a sample of Rep2CapAnc80L65_OptMin, 48 hours post-infection; Lane 3 contains a sample of Rep2CapAnc80L65_OptMin, 72 hours post-infection; Lane 4 contains a sample of Rep2CapAnc80L65_IntronMin, 24 hours post-infection; Lane 5 contains a Rep2CapAnc80L65_IntronMin, 48 hours post-infection; Lane 6 contains a Rep2CapAnc80L65_IntronMin, 72 hours post-infection; Lane 7 contains a sample from uninfected cells; and Lane 8 contains a control sample (Rep2cap2 40 hours post-infection).

It is believed that the significant production of AAV-Anc80L65 VP1, in both the Sf9 cells infected with a baculovirus vector comprising the OptMin construct and the Sf9 cells infected with a baculovirus vector comprising the IntronMin, substantially contribute to the good infectivity properties of the resulting AAV-Anc80L65 particles.

Further, as shown in Table 1 and Table 2 below, the recombinant Sf9 cells, that are either infected with an OptMin-containing baculovirus or an InteronMin baculovirus, produce high titers of recombinant AAV Anc80L65 virus particles.

TABLE 1
Recombinant AAV-Anc80L65 Virus Particles Produced by SfP
Cells Infected with an OptMin-Containing Baculovirus

| | | Titer qPCR | Titer qPCR |
|---|---|---|---|
| | Titer CSA | « BAC » | « REP52 » |
| No Batch | (IU/ml) | (copies/mL) | (copies/ml) |
| BAC085-C1 | 2.22E+08 | 8.9E+09 | 1.1E+10 |
| BAC085-C2 | 5.99E+08 | 7.7E+09 | 8.6E+09 |
| BAC085-C3 | 5.30E+08 | 5.2E+09 | 6.1E+09 |
| BAC085-C4 | 3.34E+08 | 5.5E+09 | 6.3E+09 |
| BAC085-C5 | 6.67E+07 | 8.5E+09 | 9.6E+09 |

TABLE 2
Recombinant AAV-Anc80L65 Virus Particles Produced by SfP
Cells Infected with an IntronMin-Containing Baculovirus

| | | Titer qPCR | Titer qPCR |
|---|---|---|---|
| | Titer CSA | « BAC » | « REP52 » |
| No Batch | (IU/ml) | (copies/mL) | (copies/ml) |
| BAC086-C1 | 2.26E+8 | 1.0E+10 | 1.1E+10 |
| BAC086-C2 | 3.41E+8 | 1.0E+10 | 1.0E+10 |
| BAC086-C3 | 8.4E+07 | 2.1E+09 | 1.5E+09 |
| BAC086-C4 | 3.54E+08 | 9.3E+09 | 1.0E+10 |
| BAC086-C5 | 1.54E+08 | 2.8E+09 | <LOD |

Still further, it was shown that both the Sf9 cells infected with a baculovirus vector comprising the OptMin construct (FIG. 4) and the Sf9 cells infected with a baculovirus vector comprising the IntronMin (FIG. 5) possess a high genetic stability.

### Example 3: Absence of a Requirement for Exogenous Assembly-Activating Protein (AAP)

Several distinct batches of recombinant Sf9 cells were prepared, respectively:
Sf9 cells infected with (i) an OptMin construct-containing baculovirus BAC090 and (ii) a transgene (GFP)-containing baculovirus BAC078, which resulting AAV particles are termed AAVBAC202;
Sf9 cells infected with (i) an OptMin construct-containing baculovirus BAC090, (ii) a transgene (GFP)-containing baculovirus BAC078 and (iii) an AAV2 AAP-expressing baculovirus BAC 080, which resulting AAV particles are termed AAVBAC203;
Sf9 cells infected with (i) an IntronMin construct-containing baculovirus BAC091 and (ii) a transgene (GFP)-containing baculovirus BAC078, which resulting AAV particles are termed AAVBAC204;
Sf9 cells infected with (i) an INtronMin construct-containing baculovirus BAC091, (ii) a transgene (GFP)-containing baculovirus BAC078 and (iii) an AAV2 AAP-expressing baculovirus BAC 080, which resulting AAV particles are termed AAVBAC205;

The AAV-Anc80L65 virus particles production yields are disclosed in Tables 3-6 below.

TABLE 3
AAVAnc80L65 Yields at Harvest and After Purification by Cesium
Chloride Purification Using rep2capAnc80L65_optMin
Without Addition of AAP in trans

| No batch | | Titer (vg/mL) | Titer (vg/tot) |
|---|---|---|---|
| AAVBAC202 | Harvest | 1.8E+10 | 9E+12 |
| | Purified (Cscl) | 5.7E+11 | 1.0716E+12 |

TABLE 4
AAVAnc80L65 Yields at Harvest and After Purification by Cesium
Chloride Purification Using rep2capAnc80L65_optMin
With Addition of AAP in trans

| No batch | | Titer (vg/Ml) | Titer (vg/tot) |
|---|---|---|---|
| AAVBAC203 | Harvest | 2.3E+10 | 1.15E+13 |
| | Purified (Cscl) | 8.1E+11 | 1.944E+12 |

TABLE 5
AAVAnc80L65 Yields at Harvest and After Purification by Cesium
Chloride Purification Using rep2capAnc80L65_IntronMin
Without Addition of AAP in trans

| No batch | | Titer (vg/Ml) | Titer (vg/tot) |
|---|---|---|---|
| AAVBAC204 | Harvest | 3.7E+10 | 1.85E+13 |
| | Purified (Cscl) | 6.0E+11 | 1.512E+12 |

TABLE 6
AAVAnc80U65 Yields at Harvest and After Purification by Cesium
Chloride Purification Using rep2capAnc80L65_IntronMin
With Addition of AAP in trans

| No batch | | Titer (vg/Ml) | Titer (vg/tot) |
|---|---|---|---|
| AAVBAC205 | Harvest | 1.6E+10 | 6.4E+12 |
| | Purified (Cscl) | 4.8E+11 | 9.888E+11 |

The comparative results depicted in Tables 3 and 4 show that the same recombinant AAV Anc80L65 virus production yields are obtained in Sf9 cells infected with a OptMin construct-containing baculovirus, irrespective of whether the AAV2 Assembly Activating protein (AAP) is produced in trans.

Further, the comparative results depicted in Tables 5 and 6 show that the same recombinant AAV Anc80L65 virus production yields are obtained in Sf9 cells infected with a OptMin construct-containing baculovirus, irrespective of whether the AAV2 Assembly Activating protein (AAP) is produced in trans.

Consequently, these results show the capAnc80L65optMin and capAnc80L65_IntroMin constructs are sufficient for AAV Anc80L65 capsid formation and production of high yields of recombinant AAV Anc80L65 virus particles without requiring a trans-complementation by an exogenous Assembly-Activating Protein (AAP).

Further, as it is shown in Tables 7 and 8 below, the AAV Anc80L65 produced in insect cells possess good infectivity properties towards a variety of cell types (measured as GFU/ml). The results depicted in Tables 7 and 8 show that the AAV Anc80L65 produced in insect cells possess good infectivity properties towards both HeLa and HEK293 cell lines

TABLE 7
Infectivity of the AAV Anc80L65 Virus
Particles Towards HeLa Cells

| | Infectuous | Genome | |
|---|---|---|---|
| | Titer | titer | Ratio |
| Sample | (GFU/mL) | (vg/mL) | (vg:GFU) |
| AAVbac 202 | 9.13E+06 | 5.7E+11 | 6.24E+04 |
| AAVbac 203 | 1.29E+07 | 8.1E+11 | 6.28E+04 |
| AAVbac 204 | 7.11E+06 | 6.0E+11 | 8.44E+04 |
| AAVbac 206 | 1.16E+07 | 4.8E+11 | 3.00E+04 |

TABLE 8
Infectivity of the AAV Anc80L65 Virus
Particles Towards HEK293 Cells

| | Infectuous | Genome | |
|---|---|---|---|
| | Titer | titer | Ratio |
| Sample | (GFU/mL) | (vg/mL) | (vg:GFU) |
| AAVbac 202 | 4.85E+07 | 5.7E+11 | 1.18E+04 |
| AAVbac 203 | 5.49E+07 | 8.1E+11 | 1.48E+04 |
| AAVbac 204 | 1.75E+07 | 6.0E+11 | 3.43E+04 |
| AAVbac 206 | 1.68E+07 | 4.8E+11 | 2.86E+04 |

### Example 4: Purification of Recombinant AAV Anc80L65 Virus Particles

The present inventors have designed a process allowing a high yield purification of recombinant AAV-Anc80L65 virus particles.

Four days post-infection (i.e., 96 h), insect cells were disrupted by adding 0.5% final concentration of Triton X-100 detergent (Merck) within the bioreactors or spinners.

Benzonase® (Merck) was added simultaneously to Triton at a final concentration of 5 U/mL and the culture was incubated at 37° C. during 2h 30 min under shaking.

The suspension was clarified by one single step of depth filtration using a filtration surface of 1/0.2 μm double layer, Borosilicate glass microfiber and mixed esters of cellulose membrane, filter (Millipore) at 90LMH

The first step of purification was performed by affinity chromatography using an AKTA Explorer 100 FPLC system (GE Healthcare Life Sciences). To this end, a XK16 column (GE Healthcare Life Sciences) was prepacked with the POROS™CaptureSelect™ AAV8 (Thermo Fisher Scientific) affinity resins. The chromatography column was pre-equilibrated with 5 column volumes (CV) of equilibration buffer PBS 1× (Lonza) and 2 L of clarified lysate (containing the AAV Anc80L65 particles) were then loaded at 15 ml/min (linear velocity 450 cm/h) to allow AAV particles to bind the antibodies. Afterwards, the column was washed with 10 CV of phosphate buffered saline. To unbind the vectors from the immuno-ligands a specific buffer with acidic conditions was used (PBS pH 2.0). Those fractions showing a chromatography peak (approximately 20 ml) were neutralized immediately with 1/10 volume of 1 M Tris-HCl pH 8,0.

The subsequent step of purification involved a tangential flow filtration step by using the automated KrosFlo® Research 2i Tangentiel Flow Filtration system (Spectrum Laboratories). A 115 cm² modified polyethersulfone membrane hollow fiber unit with 100 kDa molecular weight cut off was used for this step. The purified bulk was concentrated and buffer exchanged to dPBS with Ca/Mg and addition a 0.001% of nonionic surfactant Pluronic F-68 (Gibco, Invitrogen).

The samples were then sterile filtered with polyethersulfone (PES) syringe filter, 0.22 μM (Sartorius) and stored frozen at −80° C.

As shown in Table 9 below, performing the step of immunoafinity chromatography by eluting the bound AAV Anc80L65 virus particles at an acidic pH, and more precisely at a pH 2.0, allows reaching a high purification yield.

TABLE 9
Comparative Results for the Purification of
Recombinant AAV Anc80L65 Virus Particles

| | | Input column | | Output column | | Recov- |
|---|---|---|---|---|---|---|
| | Elution | Volume | Vector | Volume | Vector | ery |
| | conditions | (ml) | genomes | (ml) | genomes | Yield |
| Condi- | PBS | 450 | 4.4E+12 | 20 | 2.8E+12 | 63% |
| tion 1 | pH = 2.0 | | | | | |
| Condi- | Acide | 450 | 4.4E+12 | 15 | 7.0E+9 | 0.2% |
| tion 2 | Citrique | | | | | |
| | 50 mM + | | | | | |
| | 300 mM | | | | | |
| | NaCl | | | | | |
| | pH = 3.4 | | | | | |

Further, as it is shown in Table 10 below, the AAV Anc80L65 produced in insect cells with capAnc80L65_optMin and purified by the process described in this example retain a infectivity comparable to AAVAnc80L65 vectors produced in mammalian cells (HEK293) and purified by iodixanol gradients.

TABLE 10
Infectivity of the AAV Anc80L65 Vector Particles Produced in Insect Cells Compared
to AAV Anc80L65 Vector Particles Produced in Mammalian Cells (HEK293)

| | | | | Vector | |
|---|---|---|---|---|---|
| | | | Infectious | genome | |
| Production | | | titer | titer | Ratio |
| system | Purification | Sample ID | (GFU/mL) | (vg/ml) | (vg/GFU) |
| Insect cells | CsCl | AAVbac 202 | 3.88E+07 | 5.70E+11 | 1.47E+04 |
| (Sf9 cells) | | AAVbac 222 | 1.36E+07 | 1.60E+11 | 1.18E+04 |
| | Affinity | TFF 251 | 6.59E+07 | 1.20E+12 | 1.82E+04 |
| | chromatography | TFF 253 | 6.06E+07 | 4.30E+11 | 7.10E+03 |
| | | TFF 256 | 6.87E+07 | 6.80E+11 | 9.90E+03 |
| Mammalian | iodixanol | Bactrans029 | 7.27E+07 | 8.00E+11 | 1.10E+04 |
| cells | | Bactrans029b | 1.21E+08 | 1.20E+12 | 9.90E+03 |
| (HEK293) | | | | | |

Listing of Sequences

| SEQ | | |
|---|---|---|
| ID | | |
| NO. | Type | Description |
| 1 | nucleic acid | AAV Anc80L65 AAP-coding sequence |
| 2 | peptide | AAV Anc80L65 AAP protein |
| 3 | nucleic acid | OptMin construct with regulatory sequences |
| 4 | nucleic acid | IntronMin construct with regulatory sequences |
| 5 | nucleic acid | Primer |
| 6 | nucleic acid | Primer |
| 7 | nucleic acid | Probe |
| 8 | nucleic acid | Primer |
| 9 | nucleic acid | Primer |
| 10 | nucleic acid | Probe |
| 11 | nucleic acid | Primer |
| 12 | nucleic acid | Primer |
| 13 | nucleic acid | Probe |
| 14 | nucleic acid | Primer |
| 15 | nucleic acid | Primer |
| 16 | nucleic acid | Probe |
| 17 | nucleic acid | Primer |
| 18 | nucleic acid | Primer |
| 19 | nucleic acid | Probe |
| 20 | nucleic acid | Primer |
| 21 | nucleic acid | Primer |
| 22 | nucleic acid | Primer |
| 23 | nucleic acid | Primer |
| 24 | nucleic acid | Primer |
| 25 | nucleic acid | Probe |
| 26 | nucleic acid | Donor plasmid comprising the OptMin construct |
| 27 | nucleic acid | Donor plasmid comprising the IntronMin construct |
| 28 | nucleic acid | pMB-GFP transgene containing vector |
| 29 | nucleic acid | pFB-GFP transgene-containing vector |
| 30 | nucleic acid | DNA fragment P10-CapAnc80L65start OPT |
| 31 | nucleic acid | DNA fragment Pl0-CapAnc80L65start IntronP10 |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A non-naturally occurring nucleic acid molecule for production of capsids of an Adeno-Associated Virus (AAV) in insect cells, wherein the nucleic acid molecules comprise a first open reading frame encoding major capsid protein VP1 and minor capsid proteins VP2 and VP3, and a second open reading frame encoding an Assembly-Activating Protein (AAP).

2. The nucleic acid molecule of claim 1, wherein expression of the nucleic acid leads to the generation of AAV virions composed of VP1, VP2, and VP3 at a stoichiometry of between 1:1:8 and 1:1:12.

3. The nucleic acid molecule of claim 1, wherein the open reading frame encoding an Assembly-Activating Protein (AAP) is functional in insect cells and comprises a start codon selected from the group consisting of CTG, ATG, ACG, TTG, GTG, ATT, and ATA

4. The nucleic acid molecule of claim 1, wherein the open reading frame encoding an Assembly-Activating Protein (AAP) has the nucleic acid sequence shown in SEQ ID NO:1.

5. The nucleic acid molecule of claim 1, wherein the open reading frame encoding the VP1, VP2, and VP3 proteins comprises a start codon of the VP1 protein, wherein the start codon is selected from the group consisting of ACG, TTG, CTG, and GTG.

6. The nucleic acid molecule of claim 1, wherein the open reading frame encoding the VP1, VP2, and VP3 proteins comprises a start codon of the VP2 protein, wherein the start codon is selected from the group comprising ACG, TTG, CTG and GTG.

7. The nucleic acid molecule of claim 1, wherein the open reading frame encoding the VP1, VP2, and VP3 proteins comprises a synthetic intron sequence within the VP1 sequence.

8. The nucleic acid molecule of claim 7, further comprising (i) a first expression control sequence controlling the expression of the VP1 sequence and (ii) a second expression control sequence controlling the expression of the VP2 and VP3 sequences.

9. The nucleic acid molecule of claim 8, wherein the second expression control sequence controlling the expression of the VP2 and VP3 sequences is located in the intron sequence.

10. The nucleic acid molecule of claim 7, wherein the open reading frame encoding the VP1, VP2 and VP3 proteins comprises a start codon of the VP2 protein, wherein the start codon is selected from the group consisting of ACG, TTG, CTG, and GTG.

11. The nucleic acid molecule of claim 1, further comprising an expression cassette for expressing AAV Rep proteins

12. A non-naturally occurring baculovirus vector comprising a nucleic acid molecule of claim 1.

13. An non-naturally occurring insect cell comprising a nucleic acid molecule according to claim 1 or a baculovirus vector according to claim 12.

14. The insect cell of claim 13, further comprising a recombinant AAV vector genome comprising a transgene nucleic acid.

15. A method for producing AAV particles, the method comprising:
a) culturing the insect cells of claim 13; and
b) collecting the AAV particles produced by the insect cells cultured at step a).

16. The method of claim 15, further comprising:
c) purifying the AAV particles collected at step b) by immunoaffinity chromatography, wherein the chromatography support is a support onto which an anti-AAV8 antibody or an AAV8-binding fragment thereof is immobilized.

17. A method for purifying AAV capsid proteins, the method comprising performing affinity chromatography on a sample comprising AAV capsid proteins using a chromatography support to which an anti-AAV8 antibody or an AAV8-binding fragment is immobilized.

18. The method of claim 17, wherein the AAV particles comprise the AAV-Anc80L65 serotype.
